(19) United States
(12) Patent Application Publication
(10) Pub. No.: US 2020/0240996 A1
(43) Pub. Date: Jul. 30, 2020

(54) IDENTIFICATION AND USE OF BIOLOGICAL PARAMETERS FOR DIAGNOSIS AND TREATMENT MONITORING

(71) Applicant: VENN BIOSCIENCES CORPORATION, South San Francisco, CA (US)

(72) Inventors: Lieza Marie Araullo DANAN-LEON, South San Francisco, CA (US); Aldo Mario Eduardo Silva CARRASCOSO, Daly City, CA (US); Carolyn Ruth BERTOZZI, Menlo Park, CA (US); Carlito Bangeles LEBRILLA, Davis, CA (US); David SPICIARICH, South San Francisco, CA (US)

(21) Appl. No.: 16/756,572

(22) PCT Filed: Oct. 18, 2018
(86) PCT No.: PCT/US18/56574

§ 371 Date: Apr. 16, 2020

Related U.S. Application Data
(60) Provisional application No. 62/573,959, filed on Oct. 18, 2017.

(51) Int. Cl. G01N 33/68; C12Q 1/6886; G01N 33/574; G16B 20/00; G16B 40/00

(52) U.S. Cl. CPC G01N 33/6842; C12Q 1/6886; G01N 33/57415; G01N 2800/08; G16B 20/00; G16B 40/00; G01N 2800/60; G01N 33/6848

(57) ABSTRACT

Systems and methods of quantifying a glycomic parameter, a genomic parameter, a proteomic parameter, a metabolic parameter, and/or a lipidomic parameter of a biological sample; obtaining a clinical parameter associated with a subject from which the one or more biological samples originated; determining one or more relationships between one or more of: (i) one or more of the quantified glycomic parameters, genomic parameters, proteomic parameters, metabolic parameters, and lipidomic parameters, (ii) a predetermined range associated with one or more of the quantified glycomic parameters, genomic parameters, proteomic parameters, metabolic parameters, and lipidomic parameters, and (iii) an obtained clinical parameter; identifying one or more biomarkers based on one or more of the determined relationships satisfying a predetermined significance criteria; and/or determining a wellness classification state of a wellness classification, the determination of the wellness classification state determined based on the one or more identified biomarkers.

### TECHNICAL FIELD

The present disclosure is generally directed toward diagnosing and treating health conditions, and in some particular embodiments the present disclosure is directed toward novel systems and methods for associating biological parameters with, inter alia, wellness classifications, wellness states, treatment effectiveness, and wellness progression or digression.

### BACKGROUND OF THE DISCLOSURE

Timely diagnosis and treatment of health conditions is of great importance to the healthcare community. Conventional processes for arriving at conclusions as to diagnosis and treatment of health conditions are wanting in accuracy and precision. In particular, conventional methods of interpreting mass spectra obtained from biological samples are subject to intervening human error. Human inputs are often subject to bias that can taint a conclusion drawn from an interpretation of such mass spectra. Novel systems and methods are needed that provide improved reliability, accuracy, and precision in mass spectra interpretation through unbiased and continuously validated decision making in an intelligent environment.

### BRIEF DESCRIPTION OF THE DRAWINGS

FIG. 1A depicts a diagram of an example system configured to identify one or more biological parameters linked to one or more wellness classifications and predictively diagnose one or more wellness states of one or more subjects based on the biological parameters, in accordance with one or more embodiments of the present disclosure.

FIG. 1B depicts a diagram of an example system configured to quantify biological parameters using a peak integration platform, and to identify one or more of such biological parameters linked to one or more wellness classifications and predictively diagnose one or more wellness states of one or more subjects based on the biological parameters.

FIG. 1C depicts an example graphical representation of mass spectra obtained for a biological sample that may be analyzed.

FIG. 1D depicts an example graphical representation of peak waveforms that may be generated based on mass spectra obtained for a biological sample that may be analyzed.

FIG. 1E illustrates an integration of the peak waveforms depicted in FIG. 1D.

FIG. 2 depicts a flowchart of an example method of determining one or more biological parameters as one or more biomarkers.

FIG. 3 depicts a diagram of an example system configured to carry out one or more automatic non-biased deep learning operations to determine biomarkers.

FIG. 4 depicts a flowchart of an example method for carrying out automatic non-biased deep learning operation to determine biomarkers.

FIG. 5 depicts a diagram of an example system configured to carry out diagnosis of a subject for a disease based on biomarkers.

FIG. 6 depicts a plot showing example changes in immunoglobulin G (IgG) glycopeptide ratios in plasma samples from breast cancer patients versus controls.

FIG. 7 depicts two plots showing changes in IgG glycopeptide ratios in plasma samples from primary sclerosing cholangitis (PSC) and primary biliary cirrhosis (PBC) samples versus healthy donors.

FIGS. 8A-8C show example plots showing separate discriminant analysis data for IgG, IgA and IgM glycopeptides, respectively, in plasma samples from PSC and PBC samples versus healthy donors.

FIG. 9 shows an example of combined discriminant analysis data for IgG, IgA and IgM glycopeptides in plasma samples from PSC and PBC patients versus healthy donors.

### DETAILED DESCRIPTION

### Definitions

As used in the present specification, the following words and phrases are generally intended to have the meanings as set forth below, except to the extent that the context in which they are used indicates otherwise.

The term “biological sample” refers to any biological fluid, cell, tissue, organ, or any portion of any one or more of the foregoing, or any combination of any one or more of the foregoing. By way of example, a “biological sample” may include one or more: tissue section(s) obtained by biopsy; cell(s) that are placed in or adapted to tissue culture; sample(s) of saliva, tears, sputum, sweat, mucous, fecal material, gastric fluid, abdominal fluid, amniotic fluid, cyst fluid, peritoneal fluid, spinal fluid, urine, synovial fluid, whole blood, serum, plasma, pancreatic juice, breast milk, lung lavage, marrow, gastric acid, bile, synovial fluid, semen, pus, aqueous humour, transudate, and the like; and any other biological matter, or any portion or combination of any one or more of the foregoing

The term “biomarker” refers to a distinctive biological or biologically-derived indicator of one or more process(es), event(s), condition(s), or any combination of the foregoing. In general, biological indicators and biologically derived indicators are detectable, quantifiable, and/or otherwise measurable. For instance, biomarker may include one or more measurable molecules or substances arising from, associated with, or derived from a subject, the presence of which is indicative of another quality (e.g., one or more process(es), event(s), condition(s), or any combination of the foregoing). A biomarker may include any one or more biological molecules (taken alone or together), or a fragment of any one or more biological molecules (taken alone or together)—the detected presence, quantity (absolute, proportionate, relative, or otherwise), measure, or change in one or more of such presence, quantity, or measure of which can be correlated with one or more particular wellness state(s). By way of example, biomarkers may include, but are not limited to, biological molecules comprising one or more: nucleotide(s), amino acid(s), fatty acid(s), steroid(s), antibodie(s), hormone(s), peptide(s), protein(s), carbohydrate(s), and the like. Further examples may comprise one or more: glycosylated peptide fragment(s), lipoprotein(s), and the like. A biomarker may be indicative of a wellness condition, such as the presence, onset, stage or status of one or more disease(s), infection(s), syndrome(s), condition(s), or other state(s), including being at-risk of one or more disease(s), infection(s), syndrome(s), or condition(s).

The term “glycan” refers to the carbohydrate portion of a glycoconjugate, such as the carbohydrate portion of a glycopeptide, glycoprotein, glycolipid or proteoglycan.

The term “glycoform” refers to a unique primary, secondary, tertiary and quaternary structure of a protein with an attached glycan of a specific structure.

The term “glycosylated peptide fragment” refers to a glycosylated peptide (or glycopeptide) having an amino acid sequence that is the same as part (but not all) of the amino acid sequence of the glycosylated protein from which the glycosylated peptide is obtained via fragmentation, e.g., with one or more protease(s).

The term “multiple reaction monitoring mass spectrometry (MRM-MS)” refers to a highly sensitive and selective method for the targeted quantification of protein/peptide in biological samples. Unlike traditional mass spectrometry, MRM-MS is highly selective (targeted), allowing researchers to fine tune an instrument to specifically look for peptides/protein fragments of interest. MRM allows for greater sensitivity, specificity, speed and quantitation of peptides/protein fragments of interest, such as a potential biomarker. MRM-MS involves using one or more of a triple quadrupole (QQQ) mass spectrometer and a quadrupole time-of-flight (qTOF) mass spectrometer.

The term “protease” refers to an enzyme that performs proteolysis or breakdown of proteins into smaller polypeptides or amino acids. Examples of a protease include, but are not limited to, one one or more of a serine protease, threonine protease, cysteine protease, aspartate protease, glutamic acid protease, metalloprotease, asparagine peptide lyase, and any combinations of the foregoing.

The term “subject” refers to a mammal. The non-liming examples of a mammal include a human, non-human primate, mouse, rat, dog, cat, horse, or cow, and the like. Mammals other than humans can be advantageously used as subjects that represent animal models of disease, pre-disease, or a pre-disease condition. A subject can be male or female. A subject can be one who has been previously identified as having a disease or a condition, and optionally has already undergone, or is undergoing, a therapeutic intervention for the disease or condition. Alternatively, a subject can also be one who has not been previously diagnosed as having a disease or a condition. For example, a subject can be one who exhibits one or more risk factors for a disease or a condition, or a subject who does not exhibit disease risk factors, or a subject who is asymptomatic for a disease or a condition. A subject can also be one who is suffering from or at risk of developing a disease or a condition.

The term “treatment” or “treating” means any treatment of a disease or condition in a subject, such as a mammal, including: 1) preventing or protecting against the disease or condition, that is, causing the clinical symptoms not to develop; 2) inhibiting the disease or condition, that is, arresting or suppressing the development of clinical symptoms; and/or 3) relieving the disease or condition that is, causing the regression of clinical symptoms.

As used herein, the singular forms “a,” “an” and “the” include plural referents unless the context clearly dictates otherwise.

### System

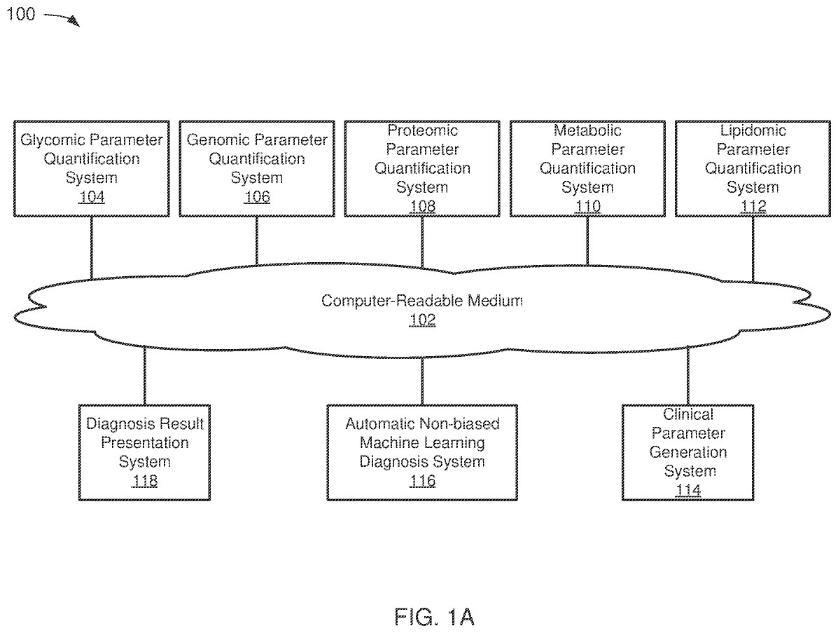

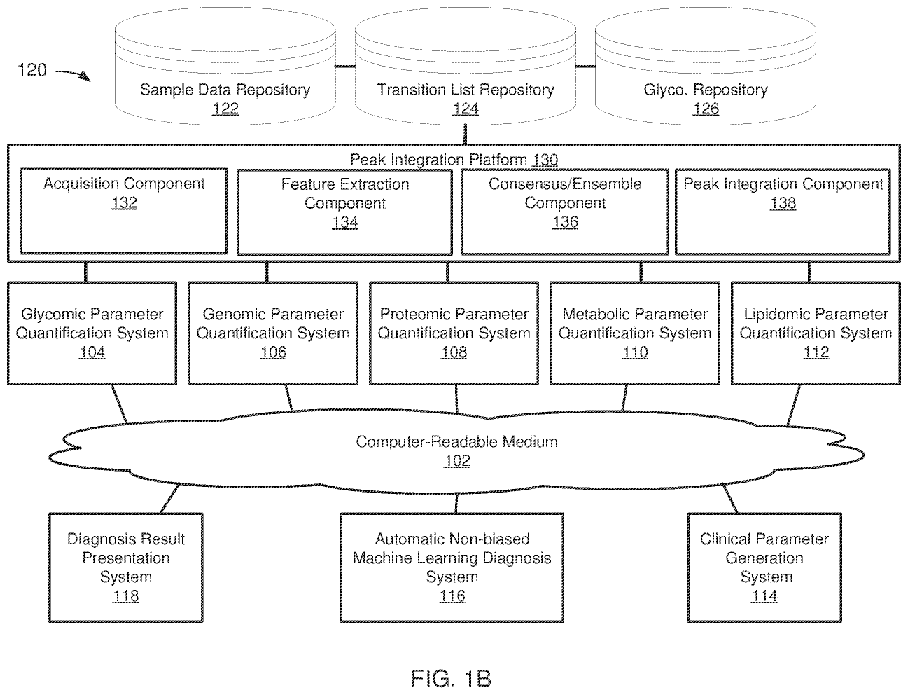

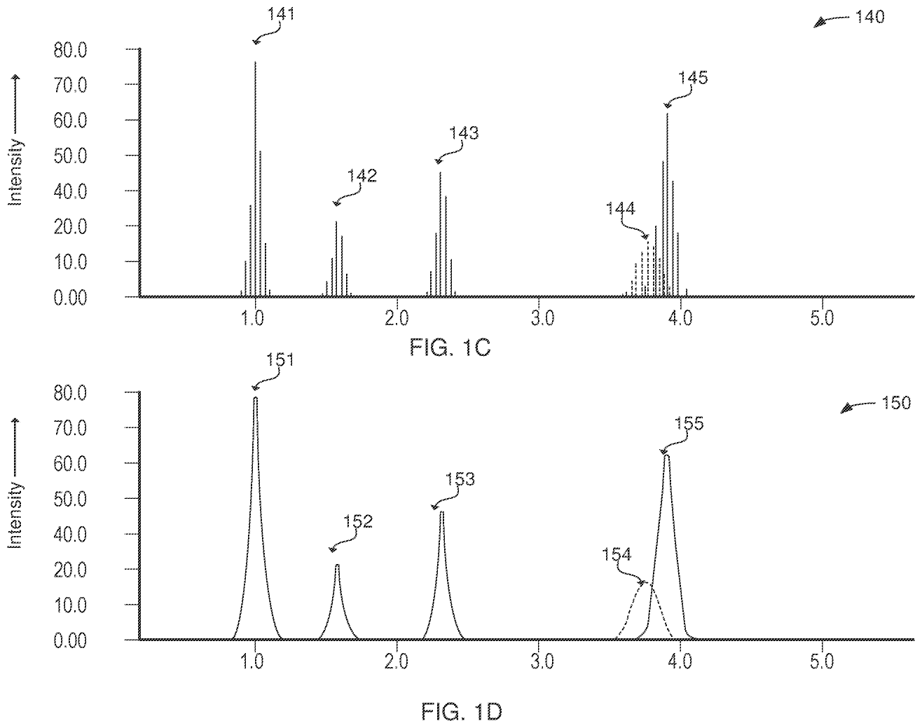

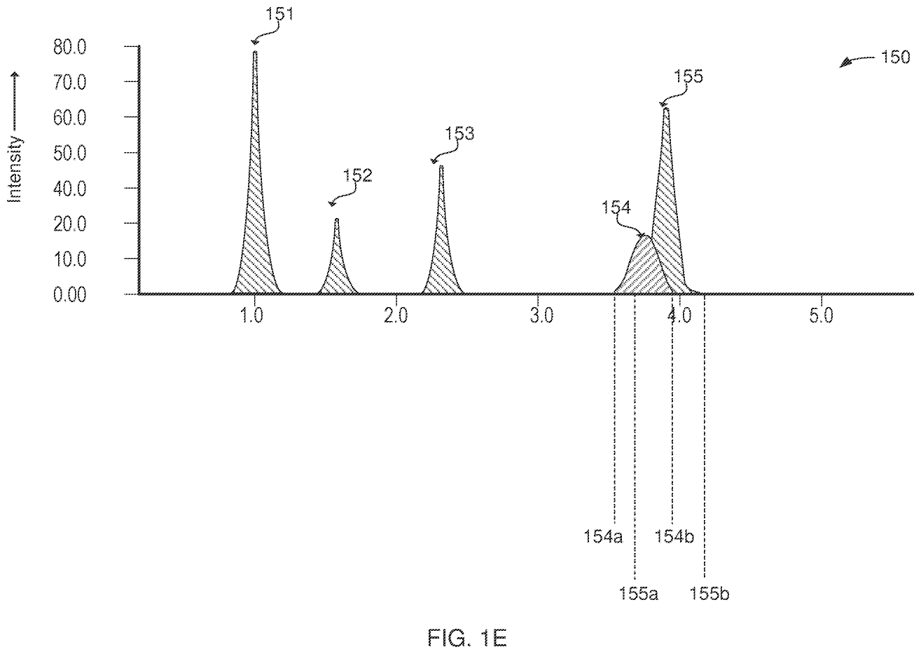

FIG. 1A depicts a diagram of an example system configured to identify biological parameters linked to wellness classifications and predictively diagnose wellness states of subjects based on the biological parameters. As shown, system 100 may include a computer-readable medium 102, a glycomic parameter quantification system 104, a genomic parameter quantification system 106, a proteomic parameter quantification system 108, a metabolic parameter quantification system 110, a lipidomic parameter quantification system 112, a clinical parameter generation system 114, an automatic non-biased machine learning diagnosis system 116, and a diagnosis result distribution system 118.

The computer-readable medium 102 is intended to represent a variety of potentially applicable technologies. For example, the computer-readable medium 102 can be used to form a network or part of a network. Where two components are co-located on a device, the computer-readable medium 102 can include a bus or other data conduit or plane. Where a first component is co-located on one device and a second component is located on a different device, the computer-readable medium 102 can include a wireless or wired back-end network or LAN. The computer-readable medium 102 can also encompass a relevant portion of a WAN or other network, if applicable.

As used in this paper, a “computer-readable medium” is intended to include all mediums that are statutory (e.g., in the United States, under 35 U.S.C. 101), and to specifically exclude all mediums that are non-statutory in nature to the extent that the exclusion is necessary for a claim that includes the computer-readable medium to be valid. Known statutory computer-readable mediums include hardware (e.g., registers, random access memory (RAM), non-volatile (NV) storage, to name a few), but may or may not be limited to hardware.

The computer-readable medium 102 or portions thereof, as well as other systems, interfaces, engines, datastores, and other devices described in this paper, can be implemented as a computer system, a plurality of computer systems, or a part of a computer system or a plurality of computer systems. In general, a computer system will include a processor, memory, non-volatile storage, and an interface. A typical computer system will usually include at least a processor, memory, and a device (e.g., a bus) coupling the memory to the processor. The processor can be, for example, a general-purpose central processing unit (CPU), such as a microprocessor, or a special-purpose processor, such as a microcontroller.

The memory can include, by way of example but not limitation, random access memory (RAM), such as dynamic RAM (DRAM) and static RAM (SRAM). The memory can be local, remote, or distributed. The bus can also couple the processor to non-volatile storage. The non-volatile storage is often a magnetic floppy or hard disk, a magnetic-optical disk, an optical disk, a read-only memory (ROM), such as a CD-ROM, EPROM, or EEPROM, a magnetic or optical card, or another form of storage for large amounts of data. Some of this data is often written, by a direct memory access process, into memory during execution of software on the computer system. The non-volatile storage can be local, remote, or distributed. The non-volatile storage is optional because systems can be created with all applicable data available in memory.

Software is typically stored in non-volatile storage. Indeed, for large programs, it may not even be possible to store the entire program in memory. Nevertheless, it should be understood that for software to run, if necessary, it is moved to a computer-readable location appropriate for processing, and for illustrative purposes, that location is referred to as the memory in this paper. Even when software is moved to the memory for execution, the processor will typically make use of hardware registers to store values associated with the software, and local cache that, ideally, serves to speed up execution. As used herein, a software program is assumed to be stored at an applicable known or convenient location (from non-volatile storage to hardware registers) when the software program is referred to as “implemented in a computer-readable storage medium.” A processor is considered to be “configured to execute a program” when at least one value associated with the program is stored in a register readable by the processor.

In one example of operation, a computer system can be controlled by operating system software, which is a software program that includes a file management system, such as a disk operating system. One example of operating system software with associated file management system software is the family of operating systems known as Windows® from Microsoft Corporation of Redmond, Wash., and their associated file management systems. Another example of operating system software with its associated file management system software is the Linux operating system and its associated file management system. The file management system causes the processor to execute the various acts required by the operating system to input and output data and to store data in the memory, including storing files on the non-volatile storage.

The bus can also couple the processor to the interface. The interface can include one or more input and/or output (I/O) devices. The I/O devices can include, by way of example but not limitation, a keyboard, a mouse or other pointing device, disk drives, printers, a scanner, and other I/O devices, including a display device. The display device can include, by way of example but not limitation, a cathode ray tube (CRT), liquid crystal display (LCD), or some other applicable known or convenient display device. The interface can include one or more of a modem or network interface. It will be appreciated that a modem or network interface can be considered to be part of the computer system. The interface can include an analog modem, ISDN modem, cable modem, token ring interface, satellite transmission interface (e.g. “direct PC”), or other interfaces for coupling a computer system to other computer systems. Interfaces enable computer systems and other devices to be coupled together in a network.

The computer systems can be compatible with or implemented as part of or through a cloud-based computing system. As used in this paper, a cloud-based computing system is a system that provides virtualized computing resources, software and/or information to client devices. The computing resources, software and/or information can be virtualized by maintaining centralized services and resources that the edge devices can access over a communication interface, such as a network. “Cloud” may be a marketing term and for the purposes of this paper can include any of the networks described herein. The cloud-based computing system can involve a subscription for services or use a utility pricing model. Users can access the protocols of the cloud-based computing system through a web browser or other container application located on their client device.

A computer system can be implemented as an engine, as part of an engine, or through multiple engines. As used in this paper, an engine includes at least two components: 1) a dedicated or shared processor and 2) hardware, firmware, and/or software modules that are executed by the processor. Depending upon implementation-specific or other considerations, an engine can be centralized or its functionality distributed. An engine can include special purpose hardware, firmware, or software embodied in a computer-readable medium for execution by the processor. The processor transforms data into new data using implemented data structures and methods, such as is described with reference to the FIGS. in this paper.

The engines described in this paper, or the engines through which the systems and devices described in this paper can be implemented, can be cloud-based engines. As used in this paper, a cloud-based engine is an engine that can run applications and/or functionalities using a cloud-based computing system. All or portions of the applications and/or functionalities can be distributed across multiple computing devices, and need not be restricted to only one computing device. In some embodiments, the cloud-based engines can execute functionalities and/or modules that end users access through a web browser or container application without having the functionalities and/or modules installed locally on the end-users' computing devices.

As used in this paper, datastores are intended to include repositories having any applicable organization of data, including tables, comma-separated values (CSV) files, traditional databases (e.g., SQL), or other applicable known or convenient organizational formats. Datastores can be implemented, for example, as software embodied in a physical computer-readable medium on a general- or specific-purpose machine, in firmware, in hardware, in a combination thereof, or in an applicable known or convenient device or system. Datastore-associated components, such as database interfaces, can be considered “part of” a datastore, part of some other system component, or a combination thereof, though the physical location and other characteristics of datastore-associated components is not critical for an understanding of the techniques described in this paper.

Datastores can include data structures. As used in this paper, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described in this paper can be cloud-based datastores. A cloud-based datastore is a datastore that is compatible with cloud-based computing systems and engines.

Referring once again to the example of FIG. 1A, the glycomic parameter quantification system 104 is coupled to the computer-readable medium 102. The glycomic parameter quantification system 104 is intended to represent an applicable system controlled to quantify glycomic parameters of biological samples and provide information about quantification results of the glycomic parameters to the computer-readable medium 102. The glycomic parameter quantification system 104 may or may not be controlled by an entity (e.g., a hospital) that collects biological samples to quantify glycomic parameters obtained from biological samples. Glycomic parameters can include an amount and change of amount of glycosylated proteins included in biological samples, an amount and change of amount of types of glycosylated peptide fragments that are fragmented from the glycosylated proteins, and a source of the biological sample. In an implementation, the glycomic parameter quantification system 104 continuously operates, such that quantification results of a new biological sample can be obtained whenever a new biological sample is obtained.

In some embodiments, biological samples are from one or more past studies that occurred over a span of 1 to 50 years or more. In some embodiments, the studies are accompanied by various other clinical parameters and previously known information such as a subject's age, height, weight, ethnicity, medical history, and the like. Such additional information can be useful in associating a subject with a wellness classification. In some embodiments, the biological samples are one or more clinical samples collected prospectively from subjects.

In one embodiment, a biological sample isolated from a subject is body tissue, saliva, tears, sputum, spinal fluid, urine, synovial fluid, whole blood, serum, or plasma. In another embodiment, a biological sample isolated from a subject is whole blood, serum, or plasma. In some embodiments, subjects are mammals. In some of those embodiments, the subjects are humans.

In one embodiment, glycosylated proteins considered for quantifying the glycomic parameters are one or more of alpha-1-acid glycoprotein, alpha-1-antitrypsin, alpha-1B-glycoprotein, alpha-2-HS-glycoprotein, alpha-2-macroglobulin, antithrombin-III, apolipoprotein B-100, apolipoprotein D, apolipoprotein F, beta-2-glycoprotein 1, ceruloplasmin, fetuin, fibrinogen, immunoglobulin (Ig) A, IgG, IgM, haptoglobin, hemopexin, histidine-rich glycoprotein, kininogen-1, serotransferrin, transferrin, vitronectin, and zinc-alpha-2-glycoprotein.

In one embodiment, glycosylated peptide fragments considered for quantifying glycomic parameters are one or more of O-glycosylated and N-glycosylated. In another embodiment, glycosylated peptide fragments considered for quantifying glycomic parameters have an average length of from 5 to 50 amino acid residues. In another embodiments, the glycosylated peptide fragments have an average length of from about 5 to about 45, or from about 5 to about 40, or from about 5 to about 35, or from about 5 to about 30, or about from 5 to about 25, or from about 5 to about 20, or from about 5 to about 15, or from about 5 to about 10, or from about 10 to about 50, or from about 10 to about 45, or from about 10 to about 40, or from about 10 to about 35, or from about 10 to about 30, or from about 10 to about 25, or from about 10 to about 20, or from about 10 to about 15, or from about 15 to about 45, or from about 15 to about 40, or from about 15 to about 35, or from about 15 to about 30, or about from 15 to about 25 or from about 15 to about 20 amino acid residues. In one embodiment, the glycosylated peptide fragments have an average length of about 15 amino acid residues. In another embodiment, the glycosylated peptide fragments have an average length of about 10 amino acid residues. In another embodiment, the glycosylated peptide fragments have an average length of about 5 amino acid residues.

In an embodiment, fragmentation of the glycosylated proteins is carried out using one or more proteases. In one embodiment, one or more of the proteases is a serine protease, threonine protease, cysteine protease, aspartate protease, glutamic acid protease, metalloprotease, asparagine peptide lyase or a combination thereof. A few representative examples of a protease include, but are not limited to, trypsin, chymotrypsin, endoproteinase, Asp-N, Arg-C, Glu-C, Lys-C, pepsin, thermolysin, ealastase, papain, proteinase K, subtilisin, clostripain, carboxypeptidase and the like. In another embodiment, the present disclosure provides the methods as described herein, wherein the one or more proteases comprise at least two proteases. In another embodiment, fragmentation and quantification of the glycosylated proteins employs liquid chromatography-mass spectrometry (LC-MS) techniques using multiple reaction monitoring mass spectrometry (MRM-MS), which enables quantification of hundreds of glycosylated peptide fragments (and their parent proteins) in a single LC/MRM-MS analysis. The advanced mass spectroscopy techniques of the present disclosure provide effective ion sources, higher resolution, faster separations and detectors with higher dynamic ranges that allow for broad untargeted measurements that also retain the benefits of targeted measurements.

The mass spectroscopy methods of the present disclosure are applicable to several glycosylated proteins at a time. For example, at least more than 50, or at least more than 60 or at least more than 70, or at least more than 80, or at least more than 90, or at least more than 100, or at least more than 110 or at least more than 120 glycosylated proteins can be analyzed at a time using the mass spectrometer.

In one embodiment, mass spectroscopy methods described in this paper employ QQQ or qTOF mass spectrometry. In another embodiment, mass spectroscopy methods described in this paper provide data with high mass accuracy of 10 ppm or better; or 5 ppm or better; or 2 ppm or better; or 1 ppm or better; or 0.5 ppm or better; or 0.2 ppm or better or 0.1 ppm or better at a resolving power of 5,000 or better; or 10,000 or better; or 25,000 or better; or 50,000 or better or 100,000 or better.

In the example of FIG. 1A, the genomic parameter quantification system 106 is coupled to the computer-readable medium 102. The genomic parameter quantification system 106 is intended to represent an applicable system controlled to quantify genomic parameters of biological samples and provide information about quantification results of the genomic parameters to the computer-readable medium 102. The genomic parameter quantification system 106 may or may not be controlled by an entity (e.g., a hospital) that collects biological samples to quantify the genomic parameters from biological samples. In an implementation, genomic parameters can include genome sequence of a DNA or RNA extracted from biological samples. Methods of DNA (RNA) sequencing is not particularly limited, and in an implementation, the methods may include Maxam-Gilbert sequencing, chain-termination methods, massively parallel signature sequencing (MPSS), polony sequencing, 454 pyrosequencing, illumina sequencing, SOLid sequencing, ion torrent semiconductor sequencing, DNA nanoball sequencing, heliscope single molecule sequencing, single molecule real time (SMRT) sequencing, nanopore DNA sequencing, tunneling current DNA sequencing, hybridization sequencing, mass spectrometry sequencing, microfluidic Sanger sequencing, RNAP sequencing, and in vitro virus high-throughput sequencing. In an implementation, the genomic parameter quantification system 106 continuously operates, in a similar manner as the glycomic parameter quantification system 104 for update of data.

In the example of FIG. 1A, the proteomic parameter quantification system 108 is coupled to the computer-readable medium 102. The proteomic parameter quantification system 108 is intended to represent an applicable system controlled to quantify proteomic parameters of biological samples and provide information about quantification results of the proteomic parameters to the computer-readable medium 102. The proteomic parameter quantification system 108 may or may not be controlled by an entity (e.g., a hospital) that collects biological samples to quantify the proteomic parameters from biological samples. In an implementation, proteomic parameters can include amount and change of the amount of each kind of protein included in biological samples and the source of the biological samples. Methods of detecting and/or quantifying proteins are not particularly limited, and in an implementation, the methods may include an enzyme-linked immunosorbent assay (ELISA), Western blot, Edman degradation, matrix-assisted laser desorption/ionization (MALDI), electrospray ionization (ESI), mass spectrometric immunoassay (MSIA), and stable isotope standard capture with anti-peptide antibodies method (SISCAPA). In an implementation, the proteomic parameter quantification system 108 continuously operates, in a similar manner as the glycomic parameter quantification system 104 for data updating.

In the example of FIG. 1A, the metabolic parameter quantification system 110 is coupled to the computer-readable medium 102. The metabolic parameter quantification system 110 is intended to represent an applicable system controlled to quantify metabolic parameters of biological samples and provide information about quantification results of the metabolic parameters to the computer-readable medium 102. The metabolic parameter quantification system 110 may or may not be controlled by an entity (e.g., a hospital) that collects biological samples to quantify the metabolic parameters from biological samples. In an implementation, metabolic parameters can include an amount and change of the amount of any products and/or byproducts caused by metabolism of subjects (including sugars, nucleotides, and amino acids), a biological state of subjects caused by the metabolism, a source of the biological sample, and so on. The metabolic parameters can be quantified by any know methods, e.g., Liquid chromatography-mass spectrometry (LC-MS) techniques using multiple reaction monitoring mass spectrometry (MRM-MS). In an implementation, the metabolic parameter quantification system 110 continuously operates, in a similar manner as the glycomic parameter quantification system 104 for data updating.

In the example of FIG. 1A, the lipidomic parameter quantification system 112 is coupled to the computer-readable medium 102. The lipidomic parameter quantification system 112 is intended to represent an applicable system controlled to quantify lipidomic parameters of biological samples and provide information about quantification results of the lipidomic parameters to the computer-readable medium 102. The lipidomic parameter quantification system 112 may or may not be controlled by an entity (e.g., a hospital) that collects biological samples to quantify the lipidomic parameters from biological samples. In an implementation, lipidomic parameters can include an amount and change of the amount of any lipids, including acyglycerol, wax, ceramide, phospholipid, sphingophospholipid, glycerophospholipid, sphingoglycolipid, glyceroglycolipid, lipoprotein, sulpholipid, fatty acid, terpenoid, steroid, and carotenoid, and the source of the biological sample from which the lipid was obtained. In an implementation, the lipidomic parameter quantification system 112 continuously operates, in a similar manner as the glycomic parameter quantification system 104 for data updating.

In the example of FIG. 1A, the clinical parameter generation system 114 is coupled to the computer-readable medium 102. The clinical parameter generation system 114 is intended to represent an applicable system controlled to generate clinical parameters of biological samples and provide information about the clinical parameters to the computer-readable medium 102. The clinical parameter generation system 114 may or may not be controlled by an entity (e.g., a hospital) that collects clinical data to generate the clinical parameters from subjects. In an implementation, clinical parameters can include any quantifiable and/or non-quantifiable data obtained by inspecting subjects (e.g., heart rate, blood pressure, blood type, body temperature, skin color, eye color, blood sugar concentration, weight, height, currently-perceived wellness classification state, and so on) and any data obtained by questioning subjects or obtained from medical records (e.g., life style including food, sleep and wake up time, exercise amount and frequency, smoking amount and frequency, alcoholic consumption amount and frequency, allergy, medicines that are taken, previously-suffered diseases, ethnicity, pain and origination of the pain, and so on). In an implementation, the clinical parameter generation system 114 continuously operates, in a similar manner as the glycomic parameter quantification system 104 for data updating.

Although a specific implementation is contained within a clinical and laboratory ecosystem, it should be understood other parameter generation systems can be utilized, including a social media parameter generation system that pulls data from social media regarding subjects, a behavioristic parameter generation system that pulls data regarding online activities from various sources, a governmental records parameter generation system that pulls publicly-available data from government-run websites, or the like. The larger the data sample size, the more disparate data can be incorporated into parameters used for wellness classification.

In the example of FIG. 1A, the automatic non-biased machine learning diagnosis system 116 is coupled to the computer-readable medium 102. The automatic non-biased machine learning diagnosis system 116 is intended to represent an applicable system controlled by an entity (e.g., a hospital) responsible for identifying one or more biologic parameters associated with particular wellness classifications. The entity may or may not be the same entity as that which controls the glycomic parameter quantification system 104, the genomic parameter quantification system 106, the proteomic parameter quantification system 108, the metabolic parameter quantification system 110, the lipidomic parameter quantification system 112, and the clinical parameter generation system 114.

In a specific implementation, the automatic non-biased machine learning diagnosis system 116 is capable of automatically determining abundance or dearth of one or more quantifiable biological parameters as biomarkers associated with a specific wellness classification and/or existence or lack of one or more non-quantifiable biological parameters as biomarkers associated with the specific wellness classification. Depending upon implementation-specific or other considerations, the biological parameter determined as a biomarker may be a scalar value or value range of a biological parameter, or a combination of two or more biological parameters (e.g., a ratio of two biological parameters, and a vector of two or more biological parameters). For example, a certain range (e.g., higher than a certain threshold, or between a lower threshold and a higher threshold) of a metabolic product indicates a wellness condition. In another example, a specific ratio or a ratio range of an amount of one type of glycopeptide to an amount of one type of lipid may indicates a wellness condition. In another example, a range of a quantifiable biological parameter over a certain threshold with a positive non-quantifiable parameter (e.g., non-smoker) may be a biomarker.

In a specific implementation, the automatic non-biased machine learning diagnosis system 116 prohibits or restricts user alteration of parameter settings for a specific data calculation process thereof, in order to ensure automatic machine calculation without human intervention (e.g., without human bias). This is because human bias tends to make it more difficult to find biomarkers of a wellness classification, when such biomarkers seem irrelevant to a human observer (e.g., scientist). In an example, in the automatic non-biased machine learning diagnosis system 116, each biological parameter that is taken into consideration by the automatic non-biased machine learning diagnosis system 116 has equal weight at least during an initial stage of the calculation. Stated in a different manner, during an initial stage of the calculation, the automatic non-biased machine learning diagnosis system 116 ignores no biological parameter. As the calculation process proceeds, the automatic non-biased machine learning diagnosis system 116 increasingly focuses on a first subset of the biological parameters as being correlated with a specific wellness classification, and less on a second subset of the biological parameters as being uncorrelated with the specific wellness classification (i.e., a noise component). Depending upon implementation-specific or other considerations, parameter setting alteration for the machine learning operation is protected through a user authentication system to ensure non-biased operation. Depending upon implementation-specific or other considerations, the machine learning is deep learning, neural network, linear discriminant analysis, quadratic discriminant analysis, support vector machine, random forest, nearest neighbor or a combination thereof.

In a specific implementation, the automatic non-biased machine learning diagnosis system 116 compares abundance or dearth of determined biomarkers associated with a wellness classification with quantification of the corresponding biological parameter obtained from a subject, to diagnose a wellness classification state (positive or negative) of the subject. For example, it is possible to determine that a subject has a disease when quantifications of biological parameters obtained from the subject falls within a specific range of the determined biomarkers.

In a specific implementation, the automatic non-biased machine learning diagnosis system 116 determines an effect of a medical treatment for a disease by comparing quantifications of biomarkers obtained from subjects who have the disease and have not received the treatment, subjects who have the disease and have received the treatment, and healthy subjects not having the disease (and not receiving the treatment). Here, the medical treatment can include, but are not limited to, exercise regimens, dietary supplementation, weight loss, surgical intervention, device implantation, and treatment with therapeutics or prophylactics used in subjects diagnosed or identified with a wellness condition. For example, it is possible to determine whether a medical treatment has a medically-favorable effect to treat a wellness condition when quantifications of biomarkers obtained from subjects receiving treatment are closer to quantifications of biomarkers obtained from healthy subjects, compared to quantifications of biomarkers obtained from the subject without the treatment. In a specific implementation, the automatic non-biased machine learning diagnosis system 116 is further capable of determining progress of medical treatment by comparing quantifications of biological parameters obtained from subjects who have the wellness classification and have not received treatment and subjects who have the wellness classification and have received treatment, and subjects who do not have the wellness classification (and are not receiving the treatment). For example, it is possible to determine treatment can be terminated when quantifications of biomarkers obtained from subjects receiving treatment approximately match quantifications of biomarkers obtained from healthy subjects. In a specific implementation, the automatic non-biased machine learning diagnosis system 116 is further capable of determining progress of wellness classification in a manner similar to determination of progress of treatment. In a specific implementation, the automatic non-biased machine learning diagnosis system 116 is further capable of determining or selecting an effective treatment from a plurality of possible treatments by comparing determined progress of the possible treatments.

In the example of FIG. 1A, the diagnosis result presentation system 118 is coupled to the computer-readable medium 102. The diagnosis result presentation system 118 is intended to represent an applicable system controlled by an entity (e.g., a web service provider) with a platform suitable for presentation of biological parameters determined by the automatic non-biased machine learning diagnosis system 116 and/or presentation of a diagnostic result generated by the automatic non-biased machine learning diagnosis system 116. The entity may or may not be the same entity as that which controls the glycomic parameter quantification system 104, the genomic parameter quantification system 106, the proteomic parameter quantification system 108, the metabolic parameter quantification system 110, the lipidomic parameter quantification system 112, the clinical parameter generation system 114, and/or the automatic non-biased machine learning diagnosis system 116.

Appropriate platforms include, by way of example but not limitation, web pages (e.g., the determined biological parameters and/or the diagnosis result could be presented as a message on a personal web page, such as an individual web page of a hospital), electronic messages (e.g., emails, text messages, voice messages), print media (e.g. a letter), and other platforms suitable for providing content to a subject.

A specific example of operation for determining biological parameters for a specific wellness classification and diagnosing a subject based on the biological parameters using a system such as is illustrated in the example of FIG. 1A is described below. The glycomic parameter quantification system 104 quantifies glycomic parameters (e.g., N-glycan) of biological samples (e.g., a blood sample) and provides information about quantification results of the glycomic parameters to the automatic non-biased machine learning diagnosis system 116. Similarly to the glycomic parameter quantification system 104, the genomic parameter quantification system 106, the proteomic parameter quantification system 108, the metabolic parameter quantification system 110, and the lipidomic parameter quantification system 112 quantify corresponding biological parameters of biological samples and provide information about quantification results to the automatic non-biased machine learning diagnosis system 116. The clinical parameter generation system 114 generates clinical parameters (e.g., positive/negative values made by subject for each questionnaire) of biological samples and provides information about the clinical parameters to the automatic non-biased machine learning diagnosis system 116.

The automatic non-biased machine learning diagnosis system 116 determines one or more biological parameters that is considered to be associated with one or more wellness classifications based on quantification results of at least one of the glycomic parameters received from the glycomic parameter quantification system 104, the genomic parameters received from the genomic parameter quantification system 106, the proteomic parameters received from the proteomic parameter quantification system 108, the metabolic parameters received from the metabolic parameter quantification system 110, and the lipidomic parameters received from the lipidomic parameter quantification system 112, and/or based on quantification and/or non-quantification results of the clinical parameters received from the clinical parameter generation system 114. Advantageously, the automatic non-biased machine learning diagnosis system 116 performs the determination of the one or more biological parameters as the biomarkers based on combination of data from two or more of the glycomic parameter quantification system 104, the genomic parameter quantification system 106, the proteomic parameter quantification system 108, the metabolic parameter quantification system 110, the lipidomic parameter quantification system 112, and the clinical parameter generation system 114, to improve accuracy of the biological parameters as the biomarkers.

In a specific implementation, the automatic non-biased machine learning diagnosis system 116 carries out diagnosis of a subject based on comparison of biological parameters with measured values or inspected state of the subject. The diagnosis result presentation system 118 carries out presentation (e.g., generation of a GUI) of biological parameters determined by the automatic non-biased machine learning diagnosis system 116 and/or presentation (e.g., generation of a GUI) of a diagnostic result (e.g., positive or negative) generated by the automatic non-biased machine learning diagnosis system 116.

To quantify respective biological parameters (e.g., glycomic parameters, genomic parameters, proteomic parameters, metabolic parameters, lipidomic parameters), system 100 may perform one or more quantification operations in connection with the universe of mass spectral data obtained from the mass spectrometry technologies utilized in a given embodiment of the present disclosure. In some embodiments, for example, may utilize one or more peak picking tools and related integration methods to quantify one or more respective biological parameters within a biological sample or set of biological samples. In some embodiments, a system of the present disclosure such as System 100 may be equipped with a subsystem or platform that one or more of systems 104-112 may leverage in performing quantification. An example implementation of such an embodiment is illustrated in FIG. 1B.

FIG. 1B depicts a diagram of an example system configured to quantify biological parameters using a peak integration platform, and to identify one or more of such biological parameters linked to one or more wellness classifications and predictively diagnose one or more wellness states of one or more subjects based on the biological parameters, in accordance with one or more embodiments of the present disclosure.

As shown in FIG. 1B, system 120 may include one or more of elements 102-118 discussed above with reference to FIG. 1A, in operative communication with one or more of Peak Integration Platform 130, Sample Data Repository 122, Transition List Repository 124, and Gylcoproteomic Universe Repository 126. As shown, Peak Integration Platform may be equipped with one or more of an Acquisition Component 132, a Feature Extraction Component 134, a Consensus/Ensemble Component 136, and a Peak Integration Component 138.

Acquisition component 132 may be configured to obtain a mass spectra dataset from a source (e.g., sample data repository 122) and make such mass spectra dataset information accessible to one or more other elements of system 120, including, for example, one or more components of peak integration platform 130—such as feature extraction component 134, consensus/ensemble component 136, and peak integration component 138. Acquisition component 132 may further be configured to store copies of obtained datasets in one or more other data repositories connected thereto. Acquisition component 132 may obtain data responsive to a user prompted command, or based on an automated trigger (e.g., a preset or periodic pulling of data at a particular time and from a particular source), or on a continuous basis. For example, acquisition component 132 may receive an indication from a user (e.g., by a user making selections via a computing device) that the user desired to load a particular mass spectra dataset associated with a new biological sample from a subject under investigation. Acquisition component 132 may further be configured to make obtained datasets available for access to one or more components sequentially, simultaneously (i.e., in parallel), in series in accordance with a predefined order, or in another arrangement based on a predetermined criteria. Acquisition component 132 may be a standalone application that facilitates the download of mass spectral dataset information in a specialized manner, or it may operate in concert with another application to effectuate the same.

Feature extraction component 134 may be configured to receive mass spectra data (e.g., associated with one or more biological samples from one or more subjects) from acquisition component 132, and to extract (i.e., identify) one or more proteomic features represented within the data. To effectuate feature extraction, feature extraction component may be configured to extract peptide induced signals (i.e., peaks) from the raw mass spectral data, or from pre-processed mass spectral data. A mass spectra dataset associated with a biological sample from a subject may contain tens to thousands of spectra (corresponding to intensity information for many different mass channels corresponding to isotopes) associated with many different molecular species (e.g., different molecules). Feature extraction component 134 may be configured to analyze the mass spectra dataset to determine whether any observed spectral patterns in the dataset (e.g., observed isotope distributions, peaks, etc.) correspond to a known or unknown but statistically significant/apparent molecular species. Known spectral patterns and/or isotope distributions corresponding to known molecular species may be stored in transition list repository 124, and accessible to feature extraction component 134 during operation. For example, transition list repository 124 may include information associated with known transitions between peaks and valleys that are associated with a particular feature. Transition list repository 124 may further include predetermined peak waveforms having predetermined start and stop points for integration (start and stop points generally corresponding to the valleys on either side of a peak associated with a known feature). Because mass spectral data can often include mixtures of overlapping isotope patterns and abundant noise, feature extraction component 134 may be configured to identify combinations of overlapping individual peaks, and filter out or otherwise reduce chemical and/or detector noise in the dataset.

Feature extraction component 134 utilize a peak picking tool known in the art, such as, NITPICK, Skyline, OpenMS, DIA-Umpire, PECAN, XCMS, multiplierz, MZmine, T-Biolnfo, MASS++, mslnspect, MassSpecWavelet, MALDlquant, EigenMS, PrepMS, LC-IMS-MS-Feature-Finder, mMass, IMTBX (Ion Mobility Toolbox), Grppr (Grouper), mzDesktop, Cromwell, MapQuant, pParse, MzJava, HappyTools, Mass-UP, LIMPIC, SpiceHit, ProteinPilot, PROcess, GAGfinder, Intact Mass, JUMBO, Maltcms, SpectroDive, enviPick, findMF, PNNL PreProcessor, msXpertSuite, LCMS-2D, or Siren (Sparse Isotope RegressionN). Feature extraction component 134 may be configured to apply or enable only unbiased features of any one or more of the foregoing, disallowing human intervention in the peak picking process.

In some embodiments, feature extraction component may apply any two or more peak picking operations to a given dataset (e.g., in parallel) to obtain two or more sets of feature extraction results for the dataset. Consensus/Ensemble component 136 may be configured to obtain multiple sets of feature extraction data for a dataset from feature extraction component 134, and identify consensus or non-consensus among the multiple sets of feature extraction results, or among portions of the multiple sets of feature extraction results. Consensus may be considered on a feature by feature basis, across the dataset as a whole, or any other desired criteria desired. In some embodiments, consensus for a given extracted feature (i.e., for a given peak (and associated transitions)) may be achieved with a predetermined number, percentage, or ratio of the applied peak picking operations arrive at an identification of a same peak within a given dataset.

In some embodiments, consensus/ensemble component 136 may generate a consensus dataset comprising a single set of feature extraction results that contains data for extracted features upon which consensus was obtained across multiple peak picking operations. In some embodiments, consensus/ensemble component 136 may generate an ensemble dataset comprising a single set of feature extraction results that is representative of the extracted features for which there was substantial similarity across multiple peak picking operations. In such embodiments, consensus/ensemble component 136 may be configured to generate the ensemble dataset by combining the feature extraction results across multiple sets of feature extraction results (e.g., on a feature specific basis) using a statistical operation to define one or more characteristics of a peak (e.g., a valley, a transition, a tip of the peak, a slope of the peak waveform at a point along the waveform, etc). Such a statistical operation may include one or more of an average, a median, a weighted combination, or any other combination.

Peak integration component 138 may be configured to obtain one or more feature extraction results from one or more of feature extraction component 134 and consensus/ensemble component 136 (or another component or element of system 120), and perform an integration to determine the area under the intensity curve that defines the peak associated with a given extracted feature (e.g., a given molecule). Peak integration component 138 may employ any type of integration method—e.g., trapezoidal integration, rectangular integration, etc. The area under the intensity curve for a given feature (even a unitless area) can be said to correspond to a quantity of molecules that are associated with that feature within a biological sample under consideration. Although the systems of the present disclosure need not generate a plot or graphical representation of spectra, or peak waveforms, or any other data in order to operate, FIGS. 1C, 1D, and 1F provide example plots that illustrate some of the concepts discussed above.

FIG. 1C illustrates an example of mass spectral data that may be obtained by acquisition component 132. Feature extraction component 134 may identify patterns with these spectra as being associated with distinct features. For example, feature extraction component 134 may determine that the spectra identified generally by numeral 141 (which appear to have substantially similar mass-to-charge ratios) are associated with a first feature (e.g., a first peak); feature extraction component 134 may determine that the spectra identified generally by numeral 142 (which appear to have substantially similar mass-to-charge ratios) are associated with a second feature (e.g., a second peak); feature extraction component 134 may determine that the spectra identified generally by numeral 143 (which appear to have substantially similar mass-to-charge ratios) are associated with a third feature (e.g., a third peak); feature extraction component 134 may determine that the spectra identified generally by numeral 144 (which appear to have substantially similar mass-to-charge ratios) are associated with a fourth feature (e.g., a fourth peak), and feature extraction component 134 may determine that the spectra identified generally by numeral 145 (which appear to have substantially similar mass-to-charge ratios) are associated with a fifth feature (e.g., a fifth peak).

As may be observed from FIG. 1C, the spectra of the fourth peak 144 overlap with the spectra from the fifth peak 145. The spectra for peak 144 are depicted with dotted lined to illustrate their difference from the spectra of the fifth peak 145. As noted above, feature extraction component 134 may be configured to discriminate between the two waveforms and identify such spectral patterns as being representative of two distinct features as opposed to one. Though shown with just two features for illustrative purposes in FIG. 1C, it should be appreciated that feature extraction component can be configured and/or trained to discriminate between more than two overlapping peaks, and in particular to determine or otherwise identify the transition points between individual peaks and valleys that are associated with distinct features (to identify start and stop points for later integration).

FIG. 1D illustrates example peak waveforms defining the first peak, second peak, third peak, fourth peak, and fifth peaks associated with the features extracted from the mass spectral data represented in FIG. 1C. As shown, first peak waveform 151 in FIG. 1D corresponds to the first peak 141 in FIG. 1C, and similarly, second, third, fourth, and fifth peak waveforms 152, 153, 154, 155 in FIG. 1D correspond, respectively, to the second, third, fourth, and fifth peaks 142, 143, 144, 145 in FIG. 1C.

FIG. 1E illustrates the example peak waveforms shown in FIG. 1D, here shown with the areas under the peak waveform curves shaded to symbolically depict an example integration accomplished by peak integration component 138. As shown, the system 120 of FIG. 1B is configured to determine the start and stop points along the horizontal axis for integration. For instance, system 120 may determine that the point on the horizontal axis corresponding to 154a corresponds to a transition that should serve as the starting point for integrating the peak waveform 154, and that the point on the horizontal axis corresponding to 154b corresponds to a transition that should serve as the stopping point for the integration of the peak waveform 154. Similarly, as shown, system 120 may determine that the point on the horizontal axis corresponding to 155a corresponds to a transition that should serve as the starting point for integrating the peak waveform 155, and that the point on the horizontal axis corresponding to 155b corresponds to a transition that should serve as the stopping point for the integration of the peak waveform 155.

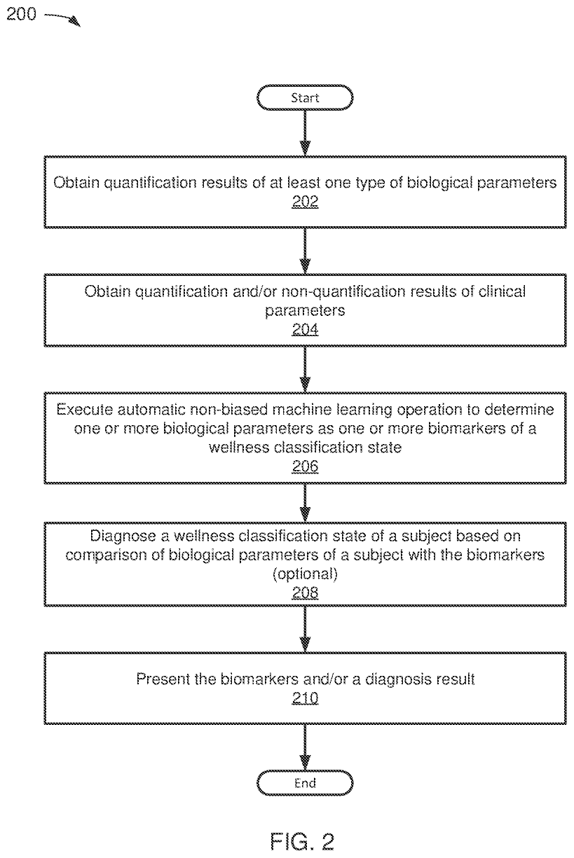

FIG. 2 depicts a flowchart 200 of an example of a method of determining one or more biological parameters as one or more biomarkers associated with one or more wellness classifications and diagnosing a subject based on the determined biomarkers. The flowchart 200 and other flowcharts in this paper are illustrated as a sequence of modules. It should be understood the sequence of the modules can be changed and the modules can be rearranged for serial or parallel processing, if appropriate.

In the example of FIG. 2, the flowchart 200 starts at module 202 with obtaining quantification results of at least one type of biological parameters. In a specific implementation, the biological parameters are obtained by analyzing biological samples. The biological parameters can include, for example, glycomic parameters, genomic parameters, proteomic parameters, metabolic parameters, and lipidomic parameters.

In the example of FIG. 2, the flowchart 200 continues to module 204 with obtaining quantification results and/or non-quantification results of clinical parameters. In a specific implementation, the results and parameters are obtained by inspecting and questioning a subject.

In the example of FIG. 2, the flowchart 200 continues to module 206 with executing automatic non-biased machine learning operation to determine one or more biological parameters as one or more biomarkers of a wellness classification. In an implementation, the automatic non-biased machine learning operation starts with equal treatment of biological and clinical parameters to remove scientific bias, and prepares no configuration for users to manually changes calculation settings of the machine learning operation.

In the example of FIG. 2, the flowchart 200 continues to module 208 with diagnosing a wellness classification state (e.g., positive or negative) of a subject based on comparison of biological parameters obtained from a biological sample of a subject with the determined biomarkers. For example, when abundance (e.g., higher than a threshold) of N-glycan and immunoglobulin G (IgG) obtained from serum are determined to be biomarkers for an ovarian cancer, it is determined whether corresponding biological parameters (i.e., N-glycan and IgG) obtained from serum of a subject are sufficiently abundant (e.g., higher than the threshold). The module 208 is optional.

In the example of FIG. 2, the flowchart 200 ends at module 210 with presenting the determined biomarkers and/or a diagnosis result, if obtained at module 208. In an implementation, the manner of presenting the diagnosis result is through a webpage presentation of the result, an email notification of the result, and/or invitation to in-person presentation at medical facilities.

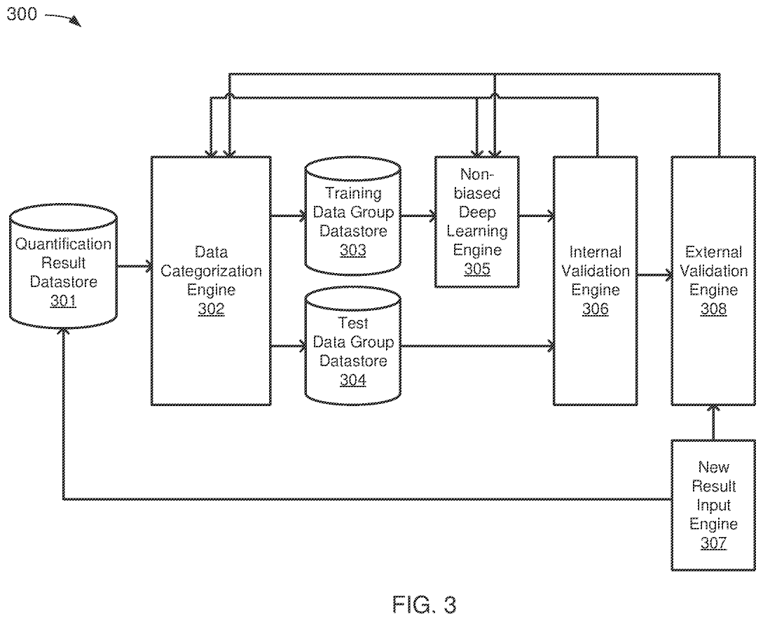

FIG. 3 depicts a diagram 300 of an example of a system for carrying out an automatic non-biased deep learning operation to determine biological parameters useful for predicting classification of subjects and optionally prediction of the classification based on candidate biological parameters. The diagram 300 includes a quantification result datastore 301, a data categorization engine 302, a training data group datastore 303, a test data group datastore 304, a non-biased deep learning engine 305, an internal validation engine 306, a new result input engine 307, and an external validation engine 308.

In the example of FIG. 3, the quantification result datastore 301 is intended to represent quantification results obtained through digitization of the biological samples, in whatever format is compatible with subsequent processing to determine candidate biological parameters for biomarkers. More specifically, for example, when the glycomic parameters are quantified, data units of the quantification result are associated with a unique identifier of a biological sample (or a subject), and include a quantification result for different kinds of glycosylated peptide fragments (e.g., known peptide fragments and/or unknown peptide fragments) in association with a parameter representing a wellness classification state (e.g., positive/negative) for one or more wellness classifications suffered or not suffered by each subject.

In the example of FIG. 3, the data categorization engine 302 is coupled to the quantification result datastore 301. The data categorization engine 302 is intended to represent specifically-purposed hardware and software that separates the quantification results in the quantification result datastore 301 into two different data groups including a training data group which is used for determining candidate biological parameters through automatic non-biased deep learning and a test data group which is used for validating the determined candidate biological parameters. The manner of sorting each data unit to one of the training and test data groups and the proportion of the training data group with respect to the test data group (training-to-test ratio) are not particularly limited, and a variety of data categorization schemes according to an algorithm can be employed.

In the example of FIG. 3, the training data group datastore 303 is coupled to the data categorization engine 302. The training data group datastore 303 is intended to represent data units categorized into the training data group by the data categorization engine 302. The data format of the data units in the training data group datastore 303 may or may not be the same as the data format of the data units in the quantification result datastore 301. In an implementation, the data units in the quantification result datastore 301 may be a non-structured data format, and the data units in the training data group datastore 303 may be a structured data format.

In the example of FIG. 3, the test data group datastore 304 is coupled to the data categorization engine 302. The test data group datastore 304 is intended to represent data units categorized into a test data group by the data categorization engine 302. Similarly to the training data group datastore 303, the data format of data units in the test data group datastore 304 may or may not be the same as the data format of data units in the quantification result datastore 301. In an implementation, data units in the quantification result datastore 301 may have a non-structured data format, and data units in the test data group datastore 304 may have a structured data format.

In the example of FIG. 3, the non-biased deep learning engine 305 is coupled to the training data group datastore 303. The non-biased deep learning engine 305 is intended to represent specifically-purposed hardware and software that carries out, according to an algorithm, a non-biased deep learning process to determine one or more biological parameters as candidates for one or more biomarkers indicating a classification (e.g., disease state) of a subject.

In an implementation, the non-biased deep learning engine 305 forms an artificial neural network (ANN) comprising an input layer, an output layer, and one or more hidden layers formed between the input layer and the output layer. The input layer includes a plurality of artificial neurons, and to each of the artificial neurons of the input layer, one quantification of a part of or the whole types of glycosylated peptide fragments, and optionally further one or more parameters representing a condition of a subject, are input. Similarly, each of the one or more of the hidden layers includes a plurality of artificial neurons, and to each of the artificial neurons of each of the one or more hidden layers, one or more outputs of artificial neurons of the immediately-previous layer (e.g., the input layer or one of the hidden layers) are input. In each artificial neuron of the one or more hidden layers, inputs from the immediately-previous layer are received at certain weights according to an algorithm, and a certain calculation (e.g., XOR) is carried out. Outputs from artificial neurons of the last hidden layer of the one or more hidden layers are input to one or more artificial neurons of the output layer, and the output layer outputs one or more biological parameters as the candidate biomarkers to predict a classification (e.g., disease state). Depending upon implementation-specific or other considerations, the ANN of the non-biased deep learning engine 305 may include a neural network, such as a feedforward neural network, in which connections between layers do not form a cycle, or a recurrent neural network (RNN), in which connections between layers form a directed cycle. Depending upon implementation-specific or other considerations, a single unit of the non-biased deep learning engine 305 may perform a deep learning process for multiple wellness classifications of interest. In an alternative, a separate unit of the non-biased deep learning engine 305 may be provided for wellness classifications of interest.

In the example of FIG. 3, the internal validation engine 306 is coupled to the non-biased deep learning engine 305 and the data group datastore 304. An output of the internal validation engine 306 is also coupled to the data categorization engine 302 and the non-biased deep learning engine 305. The internal validation engine 306 is intended to represent specifically-purposed hardware and software that carries out validation of the one or more candidate biological parameters determined by the non-biased deep learning engine 305, by matching the candidate biological parameters to the data units in the test data group (in the test data group datastore 304), and output validated candidate biological parameters as biomarkers associated with a wellness classification. In a specific implementation, the internal validation engine 306 determines, with respect to each of one or more candidate biological parameters, whether a quantification of a candidate biological parameter that was obtained from a positive subject (i.e., subject having a wellness classification) included in the test data group matches abundance (or dearth) of the candidate biological parameter determined from the data units in the training data group, and whether the quantification of the candidate biological parameter that was obtained from a negative subject (i.e., subject not having the wellness classification) included in the test data group matches dearth (or abundance) of the candidate biological parameter determined from the data units in the training data group.

In a specific implementation, the matching results obtained by the internal validation engine 306 are fed back to the data categorization engine 302, and based on the matching results, the data categorization engine 302 maintains or modifies the manner of categorizing the quantification results into a training data group and a test data group. In a specific implementation, the matching results obtained by the internal validation engine 306 are fed back to the non-biased deep learning engine 305, and based on the matching results, the non-biased deep learning engine 305 maintains or modifies weights to be applied to each artificial neuron of the ANN.

In the example of FIG. 3, the new result input engine 307 is coupled to the quantification result datastore 301. The new result input engine 307 is intended to represent specifically-purposed hardware and software that inputs quantification of biological parameters of one or more new subjects (or new biological samples) into the system. New subjects may include, for example, a subject for whom a prediction diagnosis of a wellness classification based on biomarkers is to be carried out and/or a subject who has already been diagnosed as having or not having the wellness classification. Quantifications of new subjects are input to the quantification result datastore 301 as additional data units for the new subjects, and to the external validation engine 308 for prediction diagnosis of the new subjects or extended validation of biomarkers based on the quantifications of the new subjects.

In the example of FIG. 3, the external validation engine 308 is coupled to the internal validation engine 306 and the new result input engine 307. An output of the external validation engine 308 is also coupled to the data categorization engine 302 and the non-biased deep learning engine 305. In a specific implementation, the external validation engine 308 is intended to represent specifically-purposed hardware and software that carries out prediction diagnosis based on the one or more biomarkers validated by the internal validation engine 306 and/or extended validation of the one or more biomarkers, by matching the validated biomarkers to the data units of the new subjects input from the new result input engine 307. In a specific implementation, for prediction diagnosis purpose, the external validation engine 308 determines, with respect to each of one or more biomarkers, whether a quantification of a corresponding biological parameter that was obtained from positive subject matches abundance or dearth of the biomarker. In another specific implementation, for extended validation purpose, the external validation engine 308 determines, with respect to each of one or more biomarkers, whether a quantification of a biological parameter that is obtained from positive subject (i.e., subject having a wellness classification) included in the new subjects matches abundance or dearth of the biomarker, and whether the quantification of the corresponding biological parameter that was obtained from a negative subject (i.e., subject not having the wellness classification) included in the new subjects matches dearth abundance of the biomarker. Then, the external validation engine 308 outputs the validated biomarkers for presentation purpose.

In a specific implementation, similarly to the internal validation engine 306, the matching results obtained by the external validation engine 308 are fed back to the data categorization engine 302, and based on the matching results, the data categorization engine 302 maintains or modifies the manner of categorizing the quantification results into the training data group and the test data group, and/or the training-to-test ratio. In addition, the matching results obtained by the external validation engine 308 are fed back to the non-biased deep learning engine 305, and based on the matching results, the non-biased deep learning engine 305 maintains or modifies the weights to be applied to each artificial neuron of the ANN and/or other operational parameters of the deep learning to improve accuracy of determining the classification for the wellness classification.

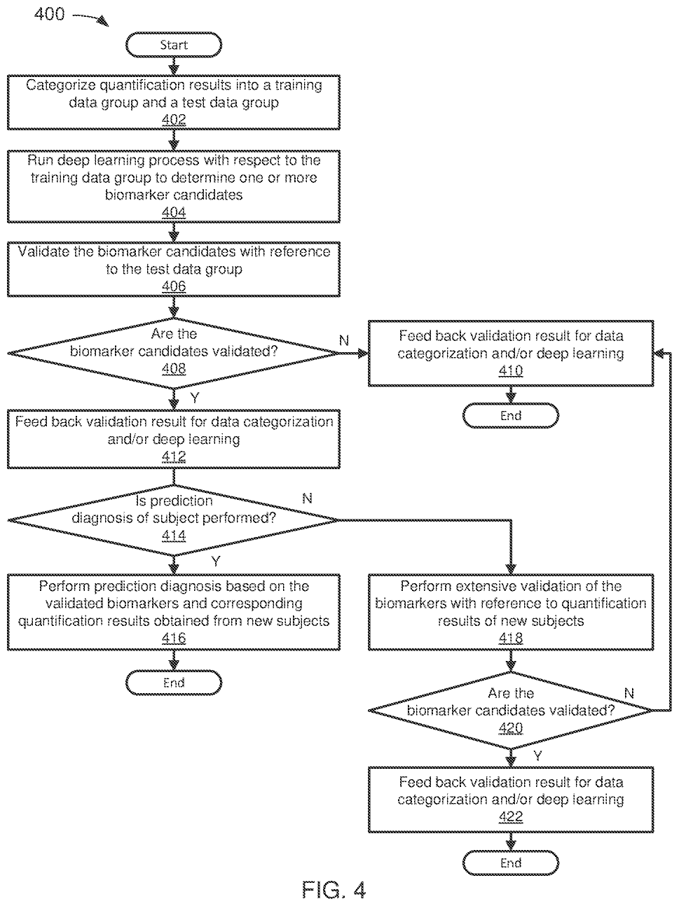

FIG. 4 depicts a flowchart 400 of an example of a method for carrying out automatic non-biased deep learning operation to determine biomarkers useful for predicting classification of subjects and prediction of the classification based on the determined biomarkers. The flowchart 400 starts at module 402 with categorizing quantification results obtained through digitization of biological samples into a training data group and a test data group.

In the example of FIG. 4, the flowchart 400 continues to module 404 where anon-biased deep learning process is executed with respect to the training data group to determine one or more biological parameters as one or more candidates for biomarkers for predicting a wellness classification.

In the example of FIG. 4, the flowchart 400 continues to module 406 where the determined candidate biological parameters are validated with reference to the test data group. In a specific implementation, validation includes determining whether a positive subject of the wellness classification has quantifications of the one or more biological parameters matching abundance or dearth of the determined candidates, and whether a negative subject of the wellness classification has quantifications of the biological parameters mismatching abundance or dearth of the determined candidates.

In the example of FIG. 4, the flowchart 400 continues to decision point 408 where it is determined that each of one or more biomarker candidates are validated. With respect to an invalidated biomarker candidate (408-N), if any, the flowchart 400 proceeds to module 410 where the validation result of the biomarker candidate for categorization of the quantification results performed at module 402 and/or the deep learning process performed at module 404 is fed back, and then the flowchart 400 ends. With respect to a validated biomarker candidate (408-Y), if any, the flowchart proceeds to module 412, where the categorization of the quantification results performed at module 402 and/or the deep learning process performed at module 404 is fed back, in a manner similar to module 410. In a specific implementation, with respect to the invalidated biomarker candidate, a neural connection between two artificial neurons may be weakened, e.g., the weight of the invalidated biomarker candidate may be decreased; and with respect to the validated biomarker candidate, a neural connection between two artificial neurons may be strengthened, e.g., the weight of the validated biomarker candidate may be increased.

In the example of FIG. 4, the flowchart 400 continues to decision point 414 where it is determined that prediction diagnosis of wellness classification is performed with respect to new subjects. If it is determined the prediction diagnosis of wellness classification is performed with respect to new subjects (414-Y), i.e., if the wellness classification state of new subjects is unknown, the flowchart 400 proceeds to module 416, where wellness classification states of the new subjects are predictively diagnosed based on comparison between abundance or dearth of the validated biomarkers (validated in module 406) and quantification results of the corresponding biological parameters obtained from biological samples of the new subjects, and then the flowchart 400 ends. For example, when abundance of a glycosylated peptide fragment over a predetermined threshold is considered to indicate a positive wellness classification state and a quantification results of the glycosylated peptide fragment obtained from a biological sample of a new subject, it is determined that the wellness classification state of the new subject is positive. In a specific implementation, invalidated biomarkers (that are invalidated in module 406) are not used for the prediction diagnosis in module 416.

If, on the other hand, it is determined the prediction diagnosis of the wellness classification is not performed with respect to new subjects (414-N), e.g., if the wellness classification state of new subjects is known, the flowchart 400 proceeds to module 418, where validated biomarkers undergo extensive validation with reference to quantification results of the new subjects. In a specific implementation, extensive validation includes determination of whether a positive subject of the wellness classification has quantifications of the one or more corresponding biological parameters matching abundance or dearth of the validated biomarkers, and whether a negative subject of the wellness classification has quantifications of the one or more corresponding biological parameters mismatching abundance or dearth of the validated biomarkers.

In the example of FIG. 4, the flowchart 400 continues to decision point 420 where it is determined each of one or more validated biomarkers are extensively validated. With respect to an invalidated biomarker (420-N), if any, the flowchart 400 returns to module 410 and continues as described previously. With respect to an extensively-validated biomarker (420-Y), if any, the flowchart 400 continues to module 422, where feedback for the categorization of the quantification results performed at module 402 and/or the deep learning process performed at module 404 is carried out, in a manner similar to module 412, and then the flowchart 400 ends. In a specific implementation, with respect to an invalidated biomarker, a neural connection between two artificial neurons may be weakened, e.g., the weight of the invalidated biomarker may be decreased; and with respect to an extensively-validated biomarker, a neural connection between two artificial neurons may be strengthened, e.g., the weight of the extensively-validated biomarker may be further increased.

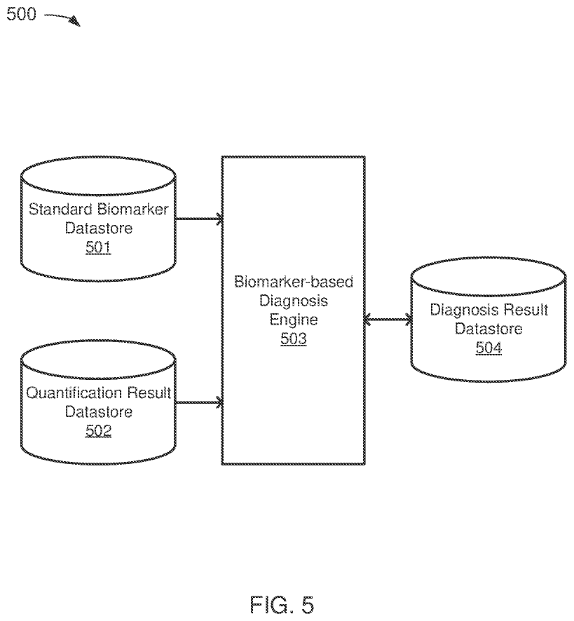

FIG. 5 depicts a diagram 500 of an example of a system for carrying out diagnosis of a subject for a wellness classification based on biomarkers determined based on a machine learning process and quantification of corresponding biological parameters of the subject obtained from biological samples of the subject. The diagram 500 includes a standard biomarker datastore 501, a quantification result datastore 502, a biomarker-based diagnosis engine 503, and a diagnosis result datastore 504.

In the example of FIG. 5, the standard biomarker datastore 501 is intended to represent details of a biomarker determined through an automatic non-biased machine learning process, for example, obtained from the internal validation engine 306 and/or the external validation engine 308 depicted in FIG. 3. For example, the details of a biomarker include that N-glycan obtained from serum higher than a first threshold and IgG higher than a second threshold indicate a positive state of a ovarian cancer. In another example, the details of a biomarker include that one type of a glycosylated peptide fragment higher than a certain threshold with a blood sugar level lower than a certain threshold indicate a positive state of a cancer. As discussed above, any single biological parameter or combination of two or more biological parameters can be a biomarker.

In the example of FIG. 5, the quantification result datastore 502 is intended to represent quantification results of quantifiable biological parameters and data of non-quantifiable biological parameters, both of which were obtained from biological samples of a subject. In an implementation, the quantification results and the data are, for example, received from one or more of the glycomic parameter quantification system 104, the genomic parameter quantification system 106, the proteomic parameter quantification system 108, the metabolic parameter quantification system 110, the lipidomic parameter quantification system 112, and the clinical parameter generation system 114 depicted in FIG. 1A.

In the example of FIG. 5, the biomarker-based diagnosis engine 503 is coupled to the standard biomarker datastore 501 and the quantification result datastore 502. In a specific implementation, the biomarker-based diagnosis engine 503 is intended to represent specifically-purposed hardware and software that carries out diagnosis of a subject based on one or more biomarker, and store results of the diagnosis in the diagnosis result datastore 504. In a specific implementation, the biomarker-based diagnosis engine 503 determines whether a subject has a wellness classification by determining whether a quantification of a biological parameter obtained from a biological sample of the subject is within a specific range based on the biomarker, and/or whether non-quantification data for a non-quantifiable parameter obtained from the subject matches the standard of the biomarker.

In a specific implementation, the biomarker-based diagnosis engine 503 determines whether a treatment applied to a subject is effective, by determining whether a quantification of a biological parameter obtained from a biological sample of the subject approaches a specific range corresponding to a healthy state, departing from another specific range corresponding to a wellness classification state, indicated by details of the biomarker, in comparison to the quantification that was obtained before the treatment was applied to the subject.

In a specific implementation, the biomarker-based diagnosis engine 503 determines an objective wellness classification progress of a subject, by determining whether a quantification of a biological parameter obtained from a biological sample of the subject increases or decreases in a specific range corresponding to a wellness classification state, departing from another specific range corresponding to a healthy state, indicated by details of the biomarker, in comparison to the quantification that was obtained previously after the subject was diagnosed as having the wellness classification. For example, after a subject was diagnosed as having a heart disease, a stage of the heart disease is objectively determined based on the biomarker level.

In a specific implementation, the biomarker-based diagnosis engine 503 determines (or selects) a treatment that is considered to be suitable for a subject having a wellness classification based on diagnosis results, in particular, treatment effectiveness results, stored in the diagnosis result datastore 504. For example, the biomarker-based diagnosis engine 503 retrieves from the diagnosis result datastore 504 treatment effectiveness results of a plurality of different treatments that have been applied to subjects having the wellness classification, and selects a best treatment from the plurality of treatments, based on the quantification results of the subject and the biomarkers.

### Diseases

The methods of the present disclosure are applicable to any disease or condition that can be detected by analyzing the biological parameters obtained from the biological samples of a subject. In some embodiments, the disease or condition is cancer. In other embodiments, the cancer is acute lymphocytic leukemia (ALL), acute myeloid leukemia (AML), adrenocortical cancer, anal cancer, bladder cancer, blood cancer, bone cancer, brain tumor, breast cancer, cancer of the female genital system, cancer of the male genital system, central nervous system lymphoma, cervical cancer, childhood rhabdomyosarcoma, childhood sarcoma, chronic lymphocytic leukemia (CLL), chronic myeloid leukemia (CML), colon and rectal cancer, colon cancer, endometrial cancer, endometrial sarcoma, esophageal cancer, eye cancer, gallbladder cancer, gastric cancer, gastrointestinal tract cancer, hairy cell leukemia, head and neck cancer, hepatocellular cancer, Hodgkin's disease, hypopharyngeal cancer, Kaposi's sarcoma, kidney cancer, laryngeal cancer, leukemia, liver cancer, lung cancer, malignant fibrous histiocytoma, malignant thymoma, melanoma, mesothelioma, multiple myeloma, myeloma, nasal cavity and paranasal sinus cancer, nasopharyngeal cancer, nervous system cancer, neuroblastoma, non-Hodgkin's lymphoma, oral cavity cancer, oropharyngeal cancer, osteosarcoma, ovarian cancer, pancreatic cancer, parathyroid cancer, penile cancer, pharyngeal cancer, pituitary tumor, plasma cell neoplasm, primary CNS lymphoma, prostate cancer, rectal cancer, respiratory system, retinoblastoma, salivary gland cancer, skin cancer, small intestine cancer, soft tissue sarcoma, stomach cancer, testicular cancer, thyroid cancer, urinary system cancer, uterine sarcoma, vaginal cancer, vascular system, Waldenstrom's macroglobulinemia, Wilms' tumor, and the like. In another embodiment, the cancer is breast cancer, cervical cancer or ovarian cancer.

In another embodiment, the disease is an autoimmune disease. In another embodiment, the autoimmune disease is acute disseminated encephalomyelitis, Addison's disease, agammaglobulinemia, age-related macular degeneration, alopecia areata, amyotrophic lateral sclerosis, ankylosing spondylitis, antiphospholipid syndrome, antisynthetase syndrome, atopic allergy, atopic dermatitis, autoimmune aplastic anemia, autoimmune cardiomyopathy, autoimmune enteropathy, autoimmune hemolytic anemia, autoimmune hepatitis, autoimmune inner ear disease, autoimmune lymphoproliferative syndrome, autoimmune peripheral neuropathy, autoimmune pancreatitis, autoimmune polyendocrine syndrome, autoimmune progesterone dermatitis, autoimmune thrombocytopenic purpura, autoimmune uticaria, autoimmune uveitis, Balo disease/Balo concentric sclerosis, Behcet's disease, Berger's disease, Bickerstaffs encephalitis, Blau syndrome, Bullous pemphigoid, cancer, Castleman's disease, celiac disease, Chagas disease, chronic inflammatory demyelinating polyneuropathy, chronic recurrent multifocal osteomyelitis, chronic obstructive pulmonary disease, Churg-Strauss syndrome, cicatricial pemphigoid, Cogan syndrome, cold agglutinin disease, complement component 2 deficiency, contact dermatitis, cranial arteritis, CREST syndrome, Crohn's disease, Cushing's syndrome, cutaneous leukocytoclastic angiitis, Dego's disease, Dercum's disease, dermatitis herpetiformis, dermatomyositis, diabetes mellitus type 1, diffuse cutaneous systemic sclerosis, Dressler's syndrome, drug-induced lupus, discoid lupus erythematosus, eczema, endometriosis, enthesitis-related arthritis, eosinophilic fasciitis, eosinophilic gastroenteritis, epidermolysis bullosa acquisita, erythema nodosum, erythroblastosis fetalis, essential mixed cryoglobulinemia, Evan's syndrome, fibrodysplasia ossificans progressive, fibrosing alveolitis, gastritis, gastrointestinal pemphigoid, glomerulonephritis, Goodpasture's syndrome, Graves' disease, Guillan-Barre syndrome, Hashimoto's encephalopathy, Hashimoto's thyroiditis, Henoch-Schonlein purpura, HIV, gestational pemphigoid, hidradenitis suppurativa, Hughes-Stovin syndrome, hypogammaglobulinemia, idiopathic inflammatory demyelinating diseases, idiopathic pulmonary fibrosis, idiopathic thrombocytopenic purpura, IgA nephropathy, inclusion body myositis, chronic inflammatory demyelinating polyneuropathy, interstitial cystitis, juvenile idiopathic arthritis, Kawasaki's disease, Lambert-Eaton myasthenic syndrome, leukocytoclastic vasculitis, lichen planus, lichen sclerosus, linear IgA disease, lupus erythematosus, Majeed syndrome, Meniere's disease, microscopic polyangiitis, mixed connective tissue disease, morphea, Mucha-Habermann disease, multiple sclerosis, myasthenia gravis, myositis, narcolepsy, neuromyelitis optica, neuromyotonia, occular cicatricial pemphigoid, opsoclonus myoclonus syndrome, Ord's thyroiditis, palindromic rheumatism, pediatric autoimmune neuropsychiatric disorders associated with streptococcus, paraneoplastic cerebellar degeneration, paroxysmal nocturnal hemoglobinuria, Parry Romberg syndrome, Parsonage-Turner syndrome, Pars planitis, pemphigus vulgaris, pernicious anemia, perivenous encephalomyelitis, POEMS syndrome, polyarteritis nodosa, polymyalgia rheumatic, polymyositis, primary biliary cirrhosis, primary sclerosing cholangitis, progressive inflammatory neuropathy, psoriasis, psoriatic arthritis, pyoderma gangrenosum, pure red cell aplasia, Rasmussen's encephalitis, Raynaud phenomenon, relapsing polychondritis, Reiter's syndrome, restless leg syndrome, retroperitoneal fibrosis, rheumatoid arthritis, rheumatic fever, sarcoidosis, schizophrenia, Schmidt syndrome, Schnitzler syndrome, scleritis, scleroderma, serum sickness, Sjogren's syndrome, spondyloarthropathy, stiff person syndrome, subacute bacterial endocarditis, Susac's syndrome, Sweet's syndrome, sympathetic ophthalmia, Takayasu's arteritis, temporal arteritis, thrombocytopenia, Tolosa-Hunt syndrome, transverse myelitis, ulcerative colitis, undifferentiated connective tissue disease, urticarial vasculitis, vasculitis, vitiligo and Wegener's granulomatosis, and the like. In another embodiment, the autoimmune disease is HIV, primary sclerosing cholangitis, primary biliary cirrhosis or psoriasis.

### EXAMPLES

### Example 1

Quantification of IgG Glycopeptides as Biomarkers for Breast Cancer

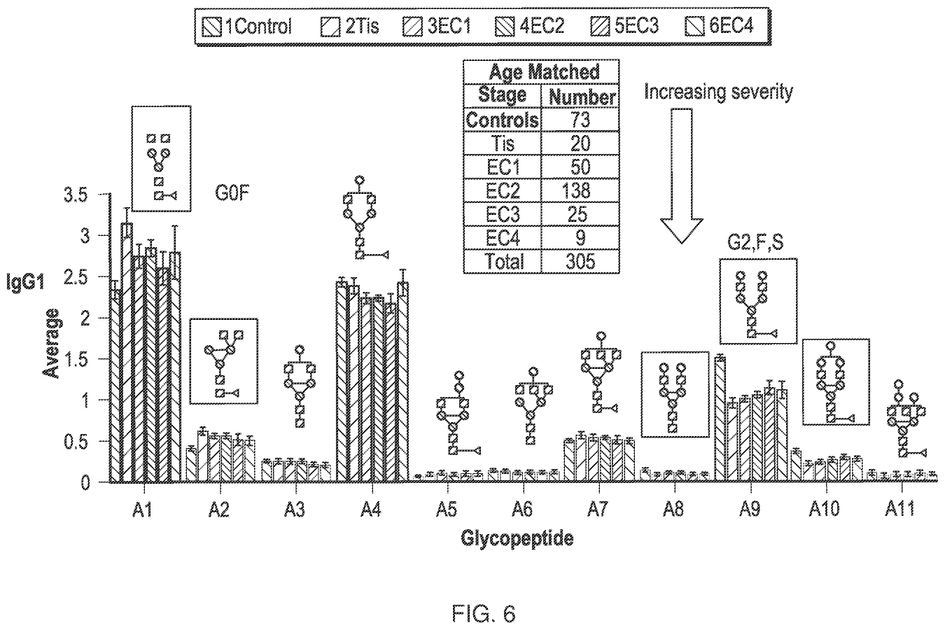

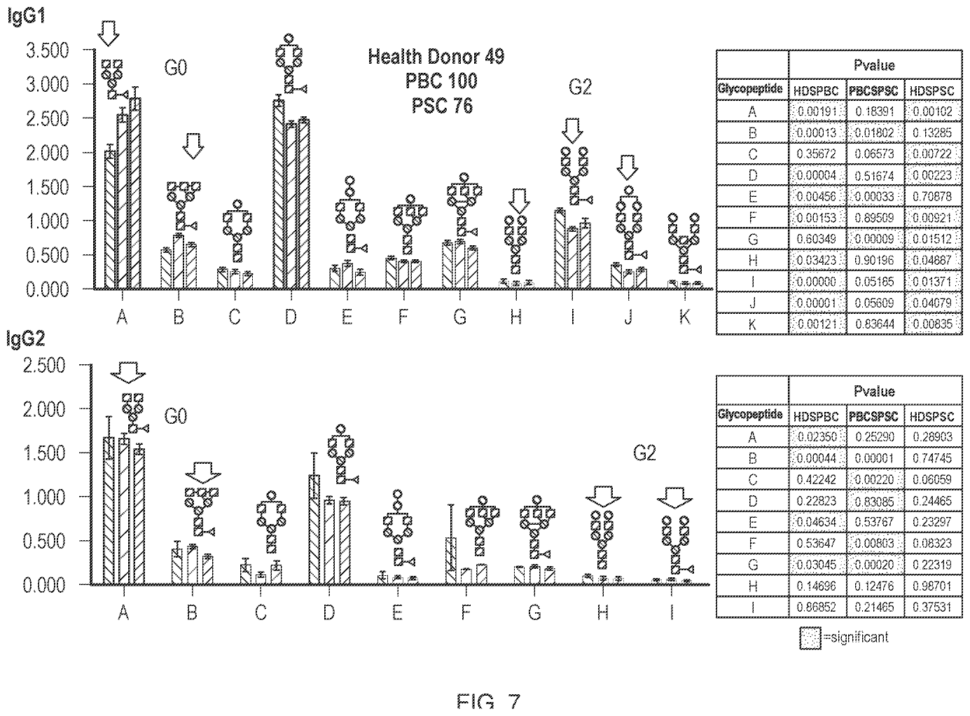

FIG. 6 shows quantification results of changes in IgG1, IgG0, and IgG2 glycopeptides in plasma samples from breast cancer patients versus controls. Plasma samples from breast cancer patients having various stages of cancer and their aged matched controls were analyzed for the IgG1, IgG0 and IgG2 glycopeptides and the changes in their ratios were compared. Specifically, 20 samples in Tis stage, 50 samples in EC1 stage, samples in EC2 stage, 25 samples in EC3 stage, 9 samples in EC4 stage and their 73 age matched control samples were subjected to MRM quantitative analysis on a QQQ mass spectrometer. As can be seen from the quantitative results in FIG. 6, the levels of certain IgG1 glycopeptides were elevated as compared to the controls, whereas the levels of certain IgG1 glycopeptides were reduced as compared to the controls in all stages of breast cancer studied in this experiment. See for example, IgG1 glycopeptides named as A1-A11, were monitored and it was found that the levels of glycopeptides A1 and A2 were elevated as compared to the control, whereas the levels of glycopeptides A8, A9, and A10 were reduced as compared to the control in all stages of breast cancer studied in this experiment. Thus, glycopeptides A1, A2, A8, A9, and A10 can be validated as biomarkers for breast cancer. It may be noted A5 appear elevated as compared to the control, albeit by a small amount, and A6 all look reduced as compared to the control, albeit by a small amount, so A5 and A6 could also be validated as biomarkers if the “small amount” were deemed adequate.

### Example 2

Quantification of IgG Glycopeptides as Potential Biomarkers for PSC and PBC

Example 2 shows quantification results of changes in IgG, IgM and IgA glycopeptides in plasma samples from patients having primary biliary cirrhosis (PBC), patients having primary sclerosing cholangitis (PSC), and healthy donors (those who do not have PBS and PSC) with reference to FIG. 7.

In Example 2, plasma samples from patients having PSC, patients having PBC and plasma samples from healthy donors were analyzed for IgG1 and IgG2 glycopeptides and the changes in their glycopeptide ratios were compared. Specifically, 100 PBC plasma samples, 76 PSC plasma samples and plasma samples from 49 healthy donors were subjected to MRM quantitative analysis on a QQQ mass spectrometer. As can be seen from the quantitative results in FIG. 7, certain IgG1 glycopeptides were elevated as compared to the healthy donors, whereas certain IgG1 glycopeptides were reduced as compared to the controls in plasma samples of patients having PBC and PSC. See for example, glycopeptide A was elevated as compared to the healthy donors in patients having PBC and PSC, whereas glycopeptides H, I, and J were reduced as compared to the healthy donors in plasma samples of patients having PBC and PSC. Thus, glycopeptides A, H, I, and J can be validated as biomarkers for PBC and PSC.

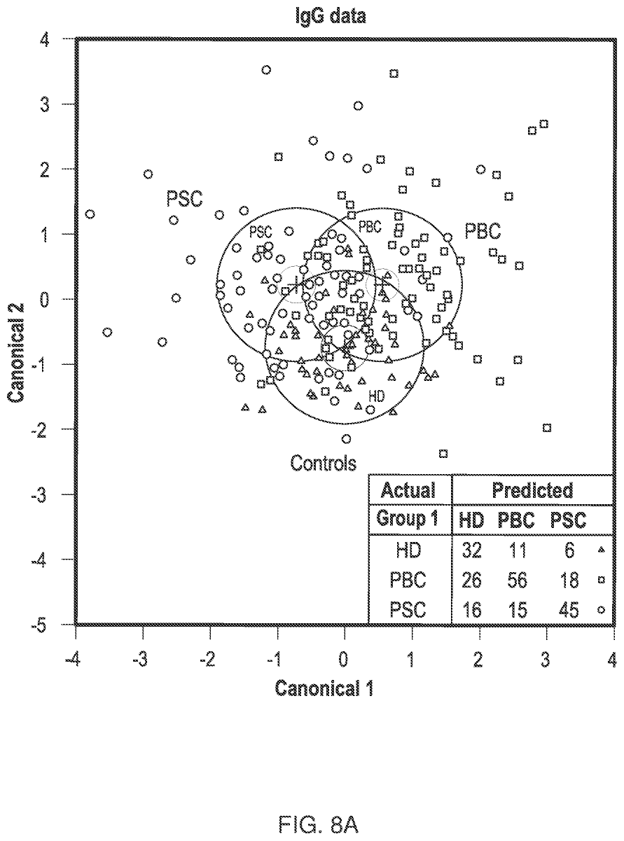

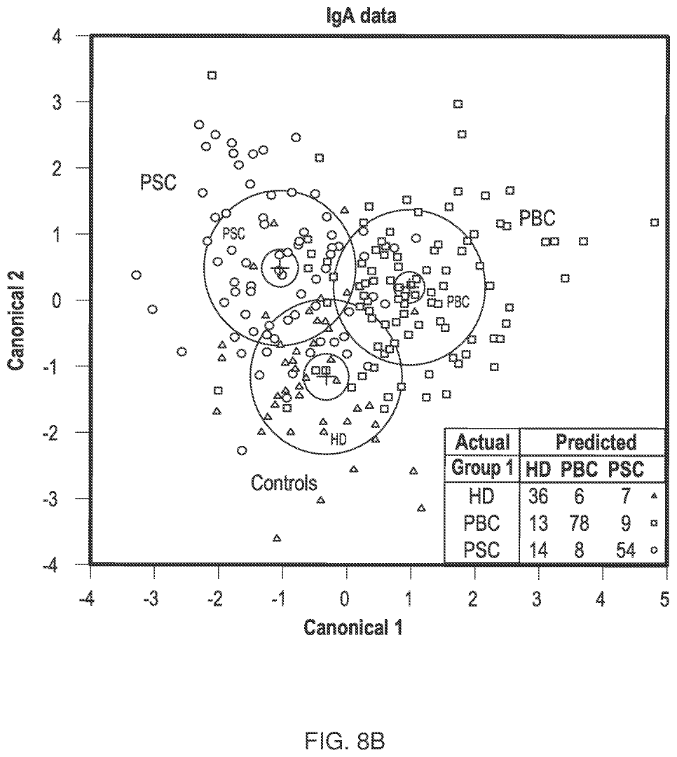

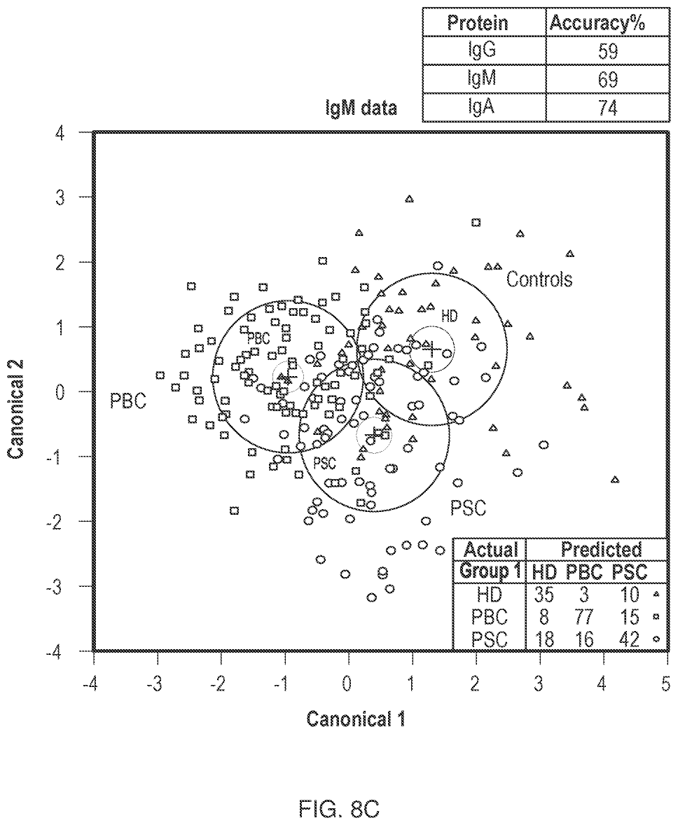

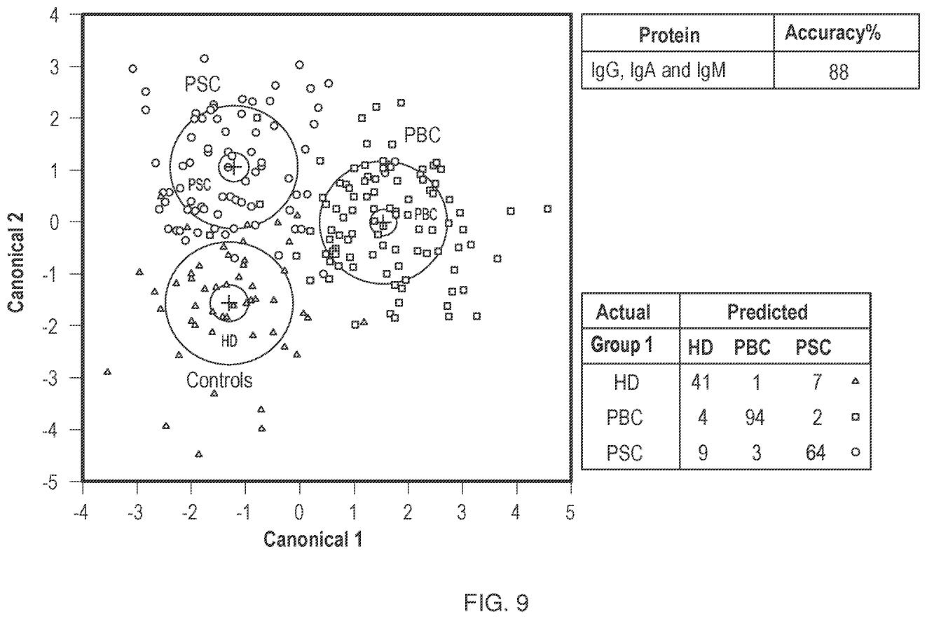

Further, a mapping of the separate and combined discriminant analysis results using a K-means clustering are shown in FIGS. 8A-8C and FIG. 9, where respectively indicate an accuracy of 88% for predicting the disease state in the combined discriminant analysis. Similar analysis was carried out on IgA and IgM glycoproteins in plasma samples of patients having PBC and plasma samples of patients having PSC. The discriminant analysis results are provided in FIGS. 8A-C which indicate the % accuracy that can be predicted based on the separate data on IgG, IgM and IgA is 59%, 69% and 74% respectively. However, when the results are combined for all IgG, IgM and IgA, the discriminant analysis provides an accuracy of about 88% as shown in FIG. 9.

These and other examples provided in this paper are intended to illustrate but not necessarily to limit the described implementation. As used herein, the term “implementation” means an implementation that serves to illustrate by way of example but not limitation. The techniques described in the preceding text and figures can be mixed and matched as circumstances demand to produce alternative implementations.

We claim:

1. A method, comprising:
isolating one or more biological samples;
quantifying one or more glycomic parameters of the one or more biological samples;
quantifying one or more genomic parameters of the one or more biological samples;
quantifying one or more proteomic parameters of the one or more biological samples;
quantifying one or more metabolic parameters of the one or more biological samples;
quantifying one or more lipidomic parameters of the one or more biological samples;
obtaining one or more clinical parameters associated with a subject from which the one or more biological samples originated;
determining one or more relationships between one or more of: (i) one or more of the quantified glycomic parameters, genomic parameters, proteomic parameters, metabolic parameters, and lipidomic parameters, (ii) a predetermined range associated with one or more of the quantified glycomic parameters, genomic parameters, proteomic parameters, metabolic parameters, and lipidomic parameters, and (iii) an obtained clinical parameter;
identifying one or more biomarkers based on one or more of the determined relationships satisfying a predetermined significance criteria;
determining a wellness classification state of a wellness classification, the determination of the wellness classification state determined based on the one or more identified biomarkers.

2. The method of claim 1, wherein: one or more of the biological samples comprises body tissue, saliva, tears, sputum, spinal fluid, urine, synovial fluid, whole blood, serum, and plasma.

3. The method of claim 1, wherein: quantifying one or more glycomic parameters comprises quantifying one or more glycosylated proteins.

4. The method of claim 3, wherein: one or more of the glycosylated proteins comprises one or more of: alpha-1-acid glycoprotein, alpha-1-antitrypsin, alpha-1B-glycoprotein, alpha-2-HS-glycoprotein, alpha-2-macroglobulin, antithrombin-III, apolipoprotein B-100, apolipoprotein D, apolipoprotein F, beta-2-glycoprotein 1, ceruloplasmin, fetuin, fibrinogen, immunoglobulin (Ig) A, IgG, IgM, haptoglobin, hemopexin, histidine-rich glycoprotein, kininogen-1, serotransferrin, transferrin, vitronectin, and zinc-alpha-2-glycoprotein.

5. The method of claim 1, wherein: quantifying one or more glycomic parameters comprises quantifying one or more glycosylated peptide fragments.

6. The method of claim 5, wherein: one or more of the glycosylated peptide fragments comprises one or more of: O-glycosylated peptide fragments and N-glycosylated peptide fragments.

7. The method of claim 5, wherein: the collection of glycosylated peptide fragments considered for quantifying the one or more glycomic parameters have an average length of about between 5 and 50 amino acid residues.

8. The method of claim 3, wherein: the glcosylated peptide fragments result from a fragmentation process applied one or more of the glycosylated proteins.

9. The method of claim 8, wherein: the fragmentation process comprises introducing at least two proteases into an environment containing the glycosylated proteins.

10. The method of claim 9, wherein: the fragmentation process comprises introducing at least two proteases into an environment containing the glycosylated proteins.

11. The method of claim 10, wherein: the at least two proteases are selected from the group consisting of: serine protease, threonine protease, cysteine protease, aspartate protease, glutamic acid protease, metalloprotease, and asparagine peptide lyase.

12. The method of claim 10, wherein: the at least two proteases are selected from the group consisting of: trypsin, chymotrypsin, endoproteinase, Asp-N, Arg-C, Glu-C, Lys-C, pepsin, thermolysin, ealastase, papain, proteinase K, subtilisin, clostripain, and carboxypeptidase.

13. The method of claim 1, wherein: quantifying one or more glycomic parameters of the one or more biological samples comprises employing a coupled chromatography procedure.

14. The method of claim 13, wherein: the coupled chromatography procedure comprises:
effectuating a liquid chromatography-mass spectrometry (LC-MS) operation.

15. The method of claim 13, wherein: the coupled chromatography procedure comprises:
effectuating a multiple reaction monitoring mass spectrometry (MRM-MS) operation.

16. The method of claim 13, wherein: the coupled chromatography procedure comprises:
effectuating a liquid chromatography-mass spectrometry (LC-MS) operation; and
effectuating a multiple reaction monitoring mass spectrometry (MRM-MS) operation.

17. The method of claim 1, wherein: quantifying one or more glycomic parameters of the one or more biological samples comprises employing one or more of a triple quadrupole (QQQ) mass spectrometry operation and a quadrupole time-of-flight (qTOF) mass spectrometry operation.

18. The method of claim 1, wherein: quantifying one or more genomic parameters comprises quantifying one or more DNA sequences within DNA isolated from the one or more biological samples.

19. The method of claim 1, wherein: quantifying one or more genomic parameters comprises quantifying one or more RNA sequences within RNA isolated from the one or more biological samples.

20. The method of claim 1, wherein: quantifying one or more proteomic parameters comprises quantifying an amount of each type of protein present in the one or more biological samples and a source of the one or more biological samples.

21. The method of claim 1, wherein: quantifying one or more proteomic parameters comprises quantifying a change in an amount of each type of protein in the one or more biological samples and a source of the one or more biological samples.

22. The method of claim 1, wherein: quantifying one or more metabolic parameters comprises quantifying a change in an amount of a byproduct attributable to the occurrence of a metabolic activity of a subject from which the biological sample originated.

23. The method of claim 22, wherein: the byproduct comprises one or more of a sugar, a nucleotide sequence, an amino acid, and a lipidome.

24. The method of claim 1, wherein: quantifying one or more metabolic parameters comprises quantifying a change in biological state of a subject from which the biological sample originated, the change in the biological state of the subject attributable to the occurrence of a metabolic activity within the subject.

25. The method of claim 1, wherein: quantifying one or more lipidomic parameters comprises quantifying an amount one or more lipids in the one or more biological samples.

26. The method of claim 1, wherein: the one or more lipids in the one or more biological samples comprises one or more of: acyglycerol, wax, ceramide, phospholipid, sphingophospholipid, glycerophospholipid, sphingoglycolipid, glyceroglycolipid, lipoprotein, sulpholipid, fatty acid, terpenoid, steroid, and carotenoid.

27. The method of claim 1, wherein: the one or more clinical parameters associated with the subject comprise one or more of: a heart rate measure of the subject, blood pressure measure of the subject, blood type measure of the subject, body temperature measure of the subject, skin color measure of the subject, eye color measure of the subject, blood sugar concentration measure of the subject, weight measure of the subject, height measure of the subject, a perceived wellness classification state of the subject, and a subject's response to a question.

28. The method of claim 1, wherein: a determined relationship comprises one or more of a specific ratio, a ratio range, an exceeded threshold, a

29. The method of claim 1, further comprising:
comparing one or more of the glycomic parameters, genomic parameters, proteomic parameters, metabolic parameters, and lipidomic parameters identified as biomarkers associated with the wellness classification state with one or more of the following:
i. one or more of glycomic parameters, genomic parameters, proteomic parameters, metabolic parameters, and lipidomic parameters quantified for one or more other subjects having the same wellness classification state, and who were treated for the wellness classification, and
ii. one or more of glycomic parameters, genomic parameters, proteomic parameters, metabolic parameters, and lipidomic parameters quantified for one or more other subjects having the same wellness classification state, and who were not treated for the wellness classification, and
iii. one or more of glycomic parameters, genomic parameters, proteomic parameters, metabolic parameters, and lipidomic parameters quantified for one or more other subjects not having the same wellness classification state.

30. The method of claim 29, further comprising:
determining an effectiveness of a medical treatment based on the comparison.

31. The method of claim 29, further comprising:
determining a stage of a wellness classification based on the comparison.

32. A system, comprising:
a memory;
a processor electrically coupled with a non-transitory computer readable medium having machine readable instructions stored thereon, which, when executed by the processor, cause the system to:
quantify one or more of a glycomic parameter, a genomic parameter, a proteomic parameter, a metabolic parameter, and a lipidomic parameter of a biological sample; wherein quantifying comprises extracting one or more features within a mass spectral dataset associated with the biological sample; determining a peak waveform associated with the one or more extracted features, and performing an integration of the peak waveform;
obtain one or more clinical parameters associated with a subject from which the one or more biological samples originated;
determine one or more relationships between one or more of: (i) one or more of the quantified glycomic parameters, genomic parameters, proteomic parameters, metabolic parameters, and lipidomic parameters, (ii) a predetermined range associated with one or more of the quantified glycomic parameters, genomic parameters, proteomic parameters, metabolic parameters, and lipidomic parameters, and (iii) an obtained clinical parameter;
identify one or more biomarkers based on one or more of the determined relationships satisfying a predetermined significance criteria; and
determine a wellness classification state of a wellness classification, the determination of the wellness classification state determined based on the one or more identified biomarkers.

33. A method, comprising:
quantifying one or more of a glycomic parameter, a genomic parameter, a proteomic parameter, a metabolic parameter, and a lipidomic parameter of a biological sample; wherein quantifying comprises extracting one or more features within a mass spectral dataset associated with the biological sample; determining a peak waveform associated with the one or more extracted features, and performing an integration of the peak waveform;
obtaining one or more clinical parameters associated with a subject from which the one or more biological samples originated;
determining one or more relationships between one or more of: (i) one or more of the quantified glycomic parameters, genomic parameters, proteomic parameters, metabolic parameters, and lipidomic parameters, (ii) a predetermined range associated with one or more of the quantified glycomic parameters, genomic parameters, proteomic parameters, metabolic parameters, and lipidomic parameters, and (iii) an obtained clinical parameter;
identifying one or more biomarkers based on one or more of the determined relationships satisfying a predetermined significance criteria;
determining a wellness classification state of a wellness classification, the determination of the wellness classification state determined based on the one or more identified biomarkers.
